(19) United States
(12) Patent Application Publication
(10) Pub. No.: US 2024/0418706 A1
(43) Pub. Date: Dec. 19, 2024

(54) QUANTIFICATION OF CIRCULATING REGENERATIVE AND ENDOTHELIAL PROGENITOR CELLS AS PREDICTORS OF HEALTH STATUS

(71) Applicant: James VELTMEYER, La Jolla, CA (US)

(72) Inventor: James VELTMEYER, La Jolla, CA (US)

(21) Appl. No.: 18/335,977

(22) Filed: Jun. 15, 2023

Related U.S. Application Data
(60) Provisional application No. 63/347,970, filed on Jun. 1, 2022.

(51) Int. Cl. G01N 33/50

(52) U.S. Cl. CPC G01N 33/5064; G01N 2800/50

(57) ABSTRACT

Disclosed are means, methods, and protocols for identifying and quantifying circulating regenerative and/or endothelial cells in a mammal in order to predict health status and risk of various pathologies. In one embodiment, determination of cardiovascular risk is performed using a blood test that assesses circulation of CD133 expressing cells, together with OCT-4 expressing cells, together with CD34/CD205 expressing cells.

### CROSS-REFERENCE TO RELATED APPLICATIONS

This application claims priority to U.S. Provisional Application No. 63/347,970, titled “Quantification of Circulating Regenerative and Endothelial Progenitor Cells as Predictors of Health Status” filed Jun. 1, 2022, which is hereby incorporated by reference herein in its entirety.

### FIELD OF THE INVENTION

The invention relates to methods of assessing cellular populations within a subject for predicting health status.

### BACKGROUND OF THE INVENTION

Blood vessel cells, called endothelial cell, their proliferation in vivo in normal, mature arterial, venous, and capillary vessels in most mammals is reported to be extremely low, if not nonexistent. In some experimental animals, such as pigs and dogs, radiolabeling studies have demonstrated 0.6-3.0% endothelial cell turnover daily with the dividing cells restricted to focal areas in certain vessels. Whether these dividing endothelial cells are unique and possess proliferative potential that is lacking in other mature endothelium remains undetermined. In marked contrast, plating of endothelial cells derived from human or animal vessels in vitro is associated initially with brisk endothelial cell proliferation. For example, human umbilical vein endothelial cells (HUVEC) and bovine aortic endothelial cells (BAEC) are two commonly studied models for in vitro analysis of endothelial cell functions. Both HUVEC and BAEC cells proliferate well initially in culture but cell division wanes with time and cells become senescent and fail to divide after 15-20 passages. It is unknown if each endothelial cell derived from the vessels possesses similar proliferative potential or if only some of the cells can divide. Angiogenesis (neoangiogenesis) is the process of new vessel formation from pre-existing vessels; this is the process reported to give rise to new vessels in adult subjects. Recently, bone marrow derived circulating endothelial progenitor cells (EPCs) have been described and these cells have also been reported to play a role in new vessel formation, at least in some experimental murine ischemic or tumor models. Conflicting evidence indicates that bone marrow derived (EPCs) do not contribute to the endothelial lining of normal arterial, venous, and capillary vessels during development and play only a minor role in neoangiogenesis. A relationship between circulating EPCs and the endothelial cells with proliferative potential that reside in normal vessels is unknown. Emerging evidence to support the use of EPCs for angiogenic therapies or as biomarkers to assess a patient's cardiovascular disease risk and progression is accumulating and is generating enthusiasm. However, there is no uniform definition of an EPC, which makes interpretation of these studies problematic and prohibits reproduction of cell types suitable for clinical use. Although a hallmark of stem and progenitor cells (e.g. hematopoietic, intestinal, neuronal) is their ability to proliferate and give rise to functional progeny, EPCs are primarily defined by the expression of selected cell surface antigens. Sole dependence on cell surface expression of molecules can be problematic because the expression may vary with the physiologic state of the cell. No assay is reported to assess the proliferative potential (an intrinsic response) in individual endothelial cells or EPCs and thus, no comparative analysis is available. Hematopoietic and endothelial progenitor cells share a number of cell surface markers in the developing yolk sac and embryo, and genetic disruption of numerous genes affects both hematopoietic and endothelial cell development. Therefore, these lineages are hypothesized to originate from a common precursor, the hemangioblast. A hierarchy of stem and progenitor cells in hematopoietic cell development is reported. Hematopoietic progenitor cells within the hierarchy are identified by their clonogenic and proliferative potential. Although genetic studies clearly show that the origin of endothelial cells is closely linked to hematopoietic cell development, evidence to support a similar hierarchy of stem and progenitor endothelial cells based on differences in proliferative potential has not been established. That is, a hierarchy of EPCs that can be discriminated by the clonogenic and proliferative potential of individual cells analogous to the hematopoietic cell system has not been reported.

The endothelium plays several functions essential for life, including: a) acting as an anticoagulated barrier between the blood stream and interior of the blood vessels; b) allowing for selective transmigration of cells into and out of the blood stream; c) regulating blood flow through controlling smooth muscle contraction/relaxation; and d) participating in tissue remodeling [1]. A key hallmark of the aging process and perhaps one of the causative factors of health decline associated with aging appears to be loss of endothelial function. Whether as a result of oxidative stress, inflammatory stress, or senescence, deficiencies in the ability of the endothelium to respond to physiological cues can alter the ability to think [2], procreate [3], see [4], and breathe [5]. Specifically, minute alterations in the ability of endothelium to respond to neurotransmitter induced nitric oxide causes profound inability to perform even simple mental functions [6, 7]. Small increases in angiogenesis in the retina as a result of injury or glucose are associated with wet macular degeneration blindness [8]. Atherosclerosis of the penile vasculature is a major cause of erectile dysfunction [9]. The pulmonary endothelium's sensitivity to insult can cause hypertension and associated progression to decreased oxygen delivery [10].

Health of the endothelium can be quantified using several methods, including assessment of the physical and mechanical features of the vessel wall, assaying for production of systemic biomarkers released by the endothelium, and quantification of ability of blood vessels to dilate in response to increased flow [11]. Of these, one of the most commonly used assays for endothelium function is the flow mediated dilation (FMD) assay. This procedure usually involves high resolution ultrasound assessment of the diameter of the superficial femoral and brachial arteries in response to reactive hyperemia induced by a cuff. The extent of dilatation response induced by the restoration of flow is compared to dilatation induced by sublingual glyceryl trinitrate. Since the dilatation induced by flow is dependent on the endothelium acting as a mechanotransducer and the dilatation induced by glyceryl trinitrate is based on smooth muscle responses, the difference in dilatation response serves as a means of quantifying one aspect of endothelial health [12, 13]. This assay has been used to show endothelial dysfunction in conditions such as healthy aging [14-16], as well as various diverse inflammatory states including renal failure [17], rheumatoid arthritis [18], Crohn's Disease [19], diabetes [20], heart failure [21], and Alzheimer's [22]. Although it is not clear whether reduction in FMD score is causative or an effect of other properties of endothelial dysfunction, it has been associated with: a) increased tendency towards thrombosis, in part by increased vWF levels [23], b) abnormal responses to injury, such as neointimal proliferation and subsequent atherosclerosis [24], and c) increased proclivity towards inflammation by basal upregulation of leukocyte adhesion molecules [25].

As part of age and disease associated endothelial dysfunction is the reduced ability of the host to generate new blood vessel [26]. This is believed to be due, at least in part, to reduction of ischemia inducible elements such as the HIF-1 alpha transcription factor which through induction of SDF-1 and VEGF secretion play a critical role in ability of endothelium to migrate and form new capillaries in ischemic tissues [27, 28]. Accordingly, if one were to understand the causes of endothelial dysfunction and develop methods of inhibiting these causes or stimulating regeneration of the endothelium, then progression of many diseases, as well as possible increase in healthy longevity may be achieved.

### Endothelial Progenitor Cells: Rejuvenators of the Vasculature

During development endothelial cells are believed to originate from a precursor cell, the hemangioblast, which is capable of giving rise to both hematopoietic and endothelial cells [29]. Classically the endothelium was viewed as a fixed structure with relatively little self renewal, however in the last two decades this concept has fundamentally been altered. The current hypothesis is that the endothelium is constantly undergoing self renewal, especially in response to stress. A key component of endothelial turnover appears to be the existence of circulating endothelial progenitor (EPC) cells that appear to be involved in repair and angiogenesis of ischemic tissues. An early study in 1963 hinted at the existence of such circulating EPC after observations of endothelial-like cells, that were non-thrombogenic and morphologically appeared similar to endothelium, were observed covering a Dacron graft that was tethered to the thoracic artery of a pig [30]. The molecular characterization of the EPC is usually credited to a 1997 paper by Asahara et al. in which human bone marrow derived VEGR-2 positive, CD34 positive monocyte-like cells were described as having ability to differentiate into endothelial cells in vitro and in vivo based on expression of CD31, eNOS, and E-selectin [31]. These studies were expanded into hindlimb ischemia in mouse and rabbit models in which increased circulation of EPC in response to ischemic insult was observed [32]. Furthermore, these studies demonstrated that cytokine-induced augmentation of EPC mobilization elicited a therapeutic angiogenic response. Using irradiated chimeric systems, it was demonstrated that ischemia-mobilized EPC derive from the bone marrow, and that these cells participate both in sprouting of pre-existing blood vessels as well as the initiation of de novo blood vessel production [33]. Subsequent to the initial phenotypic characterization by Asahara et al [31], more detailed descriptions of the human EPC were reported. For example, CD34 cells expressing the markers VEGF-receptor 2, CD133, and CXCR-4 receptor, with migrational ability to VEGF and SDF-1 has been a more refined EPC definition [34]. However there is still some controversy as to the precise phenotype of the EPC, since the term implies only ability to differentiate into endothelium. For example, both CD34+, VEGFR2+, CD133+, as well as CD34+, VEGFR2+, CD133−have been reported to act as EPC [35]. More recent studies suggest that the subpopulation lacking CD133 and CD45 are precursor EPC [36]. Other phenotypes have been ascribed to cells with EPC activity, one study demonstrated monocyte-like cells that expressing CD14, Mac-1 and the dendritic cell marker CD11c have EPC activity based on uptake of acetylated LDL and binding to the ulex-lectin [37, 38].

While the initial investigations into the biology of EPC focused around acute ischemia, it appears that in chronic conditions circulating EPC may play a role in endothelial turnover. Apolipoprotein E knockout (ApoE KO) mice are genetically predisposed to development of atherosclerosis due to inability to impaired catabolism of triglyceride-rich lipoproteins. When these mice are lethally irradiated and reconstituted with labeled bone marrow stem cells, it was found that areas of the vasculature with high endothelial turnover, which were the areas of elevated levels of sheer stress, had incorporated the majority of new endothelial cells derived from the bone marrow EPC [39]. The possibility that endogenous bone marrow derived EPC possess such a regenerative function was also tested in a therapeutic setting. Atherosclerosis is believed to initiate from endothelial injury with a proliferative neointimal response that leads to formation of plaques. When bone marrow derived EPC are administered subsequent to wire injury, a substantial reduction in neointima formation was observed [40]. The argument can obviously made that wire injury of an artery does not resemble the physiological conditions associated with plaque development. To address this, Wassmann et al [41], used ApoE KO mice that were fed a high cholesterol diet and observed reduction in endothelial function as assessed by the flow mediated dilation assay. When EPC were administered from wild-type mice restoration of endothelial responsiveness was observed.

In the context of aging, Edelman's group performed a series of interesting experiments in which 3 month old syngeneic cardiac grafts were heterotopically implanted into 18 month old recipients. Loss of graft viability, associated with poor neovascularization, was observed subsequent to transplanting, as well as subsequent to administration of 18 month old bone marrow mononuclear cells. In contrast, when 3 month old bone marrow mononuclear cells were implanted, grafts survived. Antibody depletion experiments demonstrated bone marrow derived PDGF-BB was essential in integration of the young heart cells with the old recipient vasculature [42]. These experiments suggest that young EPC or EPC-like cells have ability to integrate and interact with older vasculature. What would be interesting is to determine whether EPC could be “revitalized” ex vivo by culture conditions or transfection with therapeutic genes such as PDGF-BB.

Given animal studies suggest EPC are capable of replenishing the vasculature, and defined markers of human EPC exist, it may be possible to contemplate EPC-based therapies. Two overarching therapeutic approaches would involve utilization of exogenous EPC or mobilization of endogenous cells. Before discussing potential therapeutic interventions, we will first examine several clinical conditions in which increasing circulating EPC may play a role in response to injury.

### SUMMARY

Preferred embodiments include methods of predicting health status in a mammal comprising the steps of: a) quantifying levels of circulating endothelial progenitor cells; b) quantifying levels of circulating regenerative cells; and c) correlating said levels of circulating endothelial progenitor cells and circulating regenerative cells with inflammatory and regenerative markers in the blood.

Preferred methods include embodiments wherein said circulating endothelial progenitor cells express CD133.

Preferred methods include embodiments wherein said circulating endothelial progenitor cells express CD34.

Preferred methods include embodiments wherein said circulating endothelial progenitor cells express CD34 and CD133.

Preferred methods include embodiments wherein said circulating endothelial progenitor cells express CD133 but lack expression of CD38.

Preferred methods include embodiments wherein said circulating endothelial progenitor cells express CD133 and CD34 but lack expression of CD34.

Preferred methods include embodiments wherein said circulating endothelial progenitor cells express VEGF-receptor.

Preferred methods include embodiments wherein said circulating endothelial progenitor cells express EGF-receptor.

Preferred methods include embodiments wherein said circulating endothelial progenitor cells express VEGF-receptor and CD45.

Preferred methods include embodiments wherein said circulating endothelial progenitor cells express VEGF-receptor and CD34.

Preferred methods include embodiments wherein said circulating endothelial progenitor cells express VEGF-receptor and CD133.

Preferred methods include embodiments wherein said circulating endothelial progenitor cells express VEGF-receptor and c-met.

Preferred methods include embodiments wherein said circulating endothelial progenitor cells express VEGF-receptor and c-met.

Preferred methods include embodiments wherein said circulating endothelial progenitor cells multiply once every approximately 12-36 hours.

Preferred methods include embodiments wherein said circulating endothelial progenitor cells multiply once every approximately 17-30 hours.

Preferred methods include embodiments wherein said circulating endothelial progenitor cells multiply once every approximately 20-24 hours.

Preferred methods include embodiments wherein said circulating endothelial progenitor cells produce interleukin 1 beta at a concentration of 1-8 picograms per million cells when stimulated with 1 ng/ml of lipopolysaccharide.

Preferred methods include embodiments wherein said circulating endothelial progenitor cells produce interleukin 1 beta at a concentration of 5-7 picograms per million cells when stimulated with 1 ng/ml of lipopolysaccharide.

Preferred methods include embodiments wherein said circulating endothelial progenitor cells produce at a concentration of 7 picograms per million cells when stimulated with 1 ng/ml of lipopolysaccharide.

Preferred methods include embodiments wherein said circulating endothelial progenitor cells produce FGF-1 at a concentration of 9-88 picograms per million cells when stimulated with 1 ng/ml of lipopolysaccharide.

Preferred methods include embodiments wherein said circulating endothelial progenitor cells produce interleukin 1 beta at a concentration of 30-79 picograms per million cells when stimulated with 1 ng/ml of lipopolysaccharide.

Preferred methods include embodiments wherein said circulating endothelial progenitor cells produce interleukin 1 beta at a concentration of 40 picograms per million cells when stimulated with 1 ng/ml of lipopolysaccharide.

Preferred methods include embodiments wherein said circulating endothelial progenitor cells express IL-3 receptor.

Preferred methods include embodiments wherein said circulating endothelial progenitor cells express IL-5 receptor.

Preferred methods include embodiments wherein said circulating endothelial progenitor cells express IL-8 receptor.

Preferred methods include embodiments wherein said circulating endothelial progenitor cells express IL-10 receptor.

Preferred methods include embodiments wherein said circulating endothelial progenitor cells express IL-13 receptor.

Preferred methods include embodiments wherein said circulating endothelial progenitor cells express IL-17 receptor.

Preferred methods include embodiments wherein said circulating endothelial progenitor cells express IL-21 receptor.

Preferred methods include embodiments wherein said circulating endothelial progenitor cells express IL-33 receptor.

Preferred methods include embodiments wherein said circulating endothelial progenitor cells express interferon alpha receptor.

Preferred methods include embodiments wherein said circulating endothelial progenitor cells express interferon beta receptor.

Preferred methods include embodiments wherein said circulating endothelial progenitor cells express interferon gamma receptor.

Preferred methods include embodiments wherein said circulating endothelial progenitor cells express interferon omega receptor.

Preferred methods include embodiments wherein said circulating endothelial progenitor cells express CD11b.

Preferred methods include embodiments wherein said circulating endothelial progenitor cells express CD11c.

Preferred methods include embodiments wherein said circulating endothelial progenitor cells express CD14.

Preferred methods include embodiments wherein said circulating endothelial progenitor cells express CD40.

Preferred methods include embodiments wherein said circulating endothelial progenitor cells express CD47.

Preferred methods include embodiments wherein said circulating endothelial progenitor cells express CD35.

Preferred methods include embodiments wherein said circulating endothelial progenitor cells express DAF.

Preferred methods include embodiments wherein said circulating endothelial progenitor cells express CD73.

Preferred methods include embodiments wherein said circulating endothelial progenitor cells express CD90.

Preferred methods include embodiments wherein said circulating endothelial progenitor cells express CD105.

Preferred methods include embodiments wherein said circulating endothelial progenitor cells express aldehyde dehydrogenase.

Preferred methods include embodiments wherein said cells possess ability to inhibit an ongoing mixed lymphocyte reaction.

Preferred methods include embodiments wherein inhibition of said mixed lymphocyte reaction is quantified by assessment of proliferation of responding lymphocytes.

Preferred methods include embodiments wherein said responding lymphocytes are T cells.

Preferred methods include embodiments wherein said T cells are CD4 T cells.

Preferred methods include embodiments wherein said CD4 T cells are selected from a group comprising of: a) Th1; b) Th2; c) Th9; and d) Th17.

Preferred methods include embodiments wherein proliferation of Th1 T cells in said mixed lymphocyte reaction is inhibited by the cells of claim 46.

Preferred methods include embodiments wherein said Th1 cells express STAT4 at a higher concentration than other CD4 T cells.

Preferred methods include embodiments wherein said Th1 cells express interleukin-2 at a higher concentration than other CD4 T cells.

Preferred methods include embodiments wherein said Th1 cells express interleukin-12 at a higher concentration than other CD4 T cells.

Preferred methods include embodiments wherein said Th1 cells express interleukin-15 at a higher concentration than other CD4 T cells.

Preferred methods include embodiments wherein said Th1 cells express interleukin-18 at a higher concentration than other CD4 T cells.

Preferred methods include embodiments wherein said Th1 cells express interferon gamma at a higher concentration than other CD4 T cells.

Preferred methods include embodiments wherein proliferation of Th9 T cells in said mixed lymphocyte reaction is inhibited by the cells of claim 46.

Preferred methods include embodiments wherein said Th9 cells produce more interleukin-9 as compared to other CD4 T cells.

Preferred methods include embodiments wherein proliferation of Th17 T cells in said mixed lymphocyte reaction is inhibited by the cells of claim 46.

Preferred methods include embodiments wherein said Th17 cells express more interleukin-17 as compared to other CD4 T cells.

Preferred methods include embodiments wherein said Th17 cells express more ror-gamma as compared to other CD4 T cells.

Preferred methods include embodiments wherein said Th17 cells express more NR2F6 as compared to other CD4 T cells.

Preferred methods include embodiments wherein said Th17 cells express more interleukin-17 receptor as compared to other CD4 T cells.

Preferred methods include embodiments wherein said Th17 cells express more interleukin-6 receptor as compared to other CD4 T cells.

Preferred methods include embodiments wherein said Th17 cells express more interleukin-21 receptor as compared to other CD4 T cells.

Preferred methods include embodiments wherein said Th17 cells express more interleukin-22 receptor as compared to other CD4 T cells.

Preferred methods include embodiments wherein said Th17 cells express more interleukin-23 receptor as compared to other CD4 T cells.

Preferred methods include embodiments wherein said Th17 cells express more interleukin-27 receptor as compared to other CD4 T cells.

Preferred methods include embodiments wherein said circulating regenerative cells are stem cells.

Preferred methods include embodiments wherein said stem cells are mesenchymal stem cells.

Preferred methods include embodiments wherein said mesenchymal stem cells are bone marrow derived.

Preferred methods include embodiments wherein said mesenchymal stem cells are plastic adherent.

Preferred methods include embodiments wherein said mesenchymal stem cells possess a fibroblastoid-like morphology.

Preferred methods include embodiments wherein said mesenchymal stem cells are capable of increasing secretion of interleukin-10 in coculture with monocytes.

Preferred methods include embodiments wherein said mesenchymal stem cells are capable of increasing secretion of interleukin-4 in coculture with monocytes.

Preferred methods include embodiments wherein said mesenchymal stem cells are capable of increasing secretion of interleukin-13 in coculture with monocytes.

Preferred methods include embodiments wherein said mesenchymal stem cells are capable of increasing secretion of TGF-beta in coculture with monocytes.

Preferred methods include embodiments wherein said mesenchymal stem cells are capable of increasing secretion of PGE-2 in coculture with monocytes.

Preferred methods include embodiments wherein said mesenchymal stem cells are capable of increasing secretion of tolerogenic exosomes in coculture with monocytes.

Preferred methods include embodiments wherein said mesenchymal stem cells do not express CD14.

Preferred methods include embodiments wherein said mesenchymal stem cells do not express CD33.

Preferred methods include embodiments wherein said mesenchymal stem cells do not express CD34.

Preferred methods include embodiments wherein said mesenchymal stem cells express CD90.

Preferred methods include embodiments wherein said mesenchymal stem cells express CD105.

Preferred methods include embodiments wherein said mesenchymal stem cells express CD73.

Preferred methods include embodiments wherein said mesenchymal stem cells express CD1.

Preferred methods include embodiments wherein said mesenchymal stem cells express CD37.

Preferred methods include embodiments wherein said mesenchymal stem cells express IL-2.

Preferred methods include embodiments wherein said mesenchymal stem cells express IL-10.

Preferred methods include embodiments wherein said mesenchymal stem cells express IL-4.

Preferred methods include embodiments wherein said mesenchymal stem cells express IL-13.

Preferred methods include embodiments wherein said mesenchymal stem cells express IL-20.

Preferred methods include embodiments wherein said mesenchymal stem cells express angiopoietin.

Preferred methods include embodiments wherein said mesenchymal stem cells express PDGF-BB.

Preferred methods include embodiments wherein said mesenchymal stem cells express IGF-1.

Preferred methods include embodiments wherein said mesenchymal stem cells express FGF-1

Preferred methods include embodiments wherein said mesenchymal stem cells express FGF-2.

Preferred methods include embodiments wherein said mesenchymal stem cells express FGF-5.

Preferred methods include embodiments wherein augmentation of circulating endothelial progenitor cells, and/or circulating regenerative cells is accomplished by administration of an inhibitor of NF-kappa B.

Preferred methods include embodiments wherein said NF-kappa B inhibitor is Perrilyl alcohol.

Preferred methods include embodiments wherein said NF-kappa B inhibitor is Protein-bound polysaccharide from basidiomycetes.

Preferred methods include embodiments wherein said NF-kappa B inhibitor is Rocaglamides.

Preferred methods include embodiments wherein said NF-kappa B inhibitor is 15-deoxy-prostaglandin J(2).

Preferred methods include embodiments wherein said NF-kappa B inhibitor is Anandamide.

Preferred methods include embodiments wherein said NF-kappa B inhibitor is vestita.

Preferred methods include embodiments wherein said NF-kappa B inhibitor is Dehydroascorbic acid.

Preferred methods include embodiments wherein said NF-kappa B inhibitor is Herbimycin A.

Preferred methods include embodiments wherein said NF-kappa B inhibitor is Isorhapontigenin.

Preferred methods include embodiments wherein said NF-kappa B inhibitor is Manumycin A.

Preferred methods include embodiments wherein said NF-kappa B inhibitor is Pomegranate fruit extract.

Preferred methods include embodiments wherein said NF-kappa B inhibitor is Tetrandine.

Preferred methods include embodiments wherein said NF-kappa B inhibitor is Thienopyridine.

Preferred methods include embodiments wherein said NF-kappa B inhibitor is Acetyl-boswellic acids.

Preferred methods include embodiments wherein said NF-kappa B inhibitor is 1′-Acetoxychavicol acetate.

Preferred methods include embodiments wherein said NF-kappa B inhibitor is Apigenin.

Preferred methods include embodiments wherein said NF-kappa B inhibitor is Cardamomin.

Preferred methods include embodiments wherein said NF-kappa B inhibitor is Diosgenin.

Preferred methods include embodiments wherein said NF-kappa B inhibitor is Furonaphthoquinone.

Preferred methods include embodiments wherein said NF-kappa B inhibitor is Guggulsterone.

Preferred methods include embodiments wherein said NF-kappa B inhibitor is Falcarindol.

Preferred methods include embodiments wherein said NF-kappa B inhibitor is Honokiol.

Preferred methods include embodiments wherein said NF-kappa B inhibitor is Hypoestoxide.

Preferred methods include embodiments wherein said NF-kappa B inhibitor is Garcinone B.

Preferred methods include embodiments wherein said NF-kappa B inhibitor is Kahweol.

Preferred methods include embodiments wherein said NF-kappa B inhibitor is Kava.

Preferred methods include embodiments wherein said NF-kappa B inhibitor is mangostin.

Preferred methods include embodiments wherein said NF-kappa B inhibitor is mangostin.

Preferred methods include embodiments wherein said NF-kappa B inhibitor is N-acetylcysteine.

Preferred methods include embodiments wherein said NF-kappa B inhibitor is Piceatannol.

Preferred methods include embodiments wherein said NF-kappa B inhibitor is Plumbagin.

Preferred methods include embodiments wherein said NF-kappa B inhibitor is Quercetin.

Preferred methods include embodiments wherein said NF-kappa B inhibitor is Rosmarinic acid.

Preferred methods include embodiments wherein said NF-kappa B inhibitor is Staurosporine.

Preferred methods include embodiments wherein said NF-kappa B inhibitor is Sulforaphane.

Preferred methods include embodiments wherein said NF-kappa B inhibitor is phenylisothiocyanate.

Preferred methods include embodiments wherein said NF-kappa B inhibitor is Theaflavin.

Preferred methods include embodiments wherein said NF-kappa B inhibitor is Tilianin.

Preferred methods include embodiments wherein said NF-kappa B inhibitor is Tocotrienol.

Preferred methods include embodiments wherein said NF-kappa B inhibitor is Wedelolactone.

Preferred methods include embodiments wherein said NF-kappa B inhibitor is Withanolides.

Preferred methods include embodiments wherein said NF-kappa B inhibitor is Zerumbone.

Preferred methods include embodiments wherein said NF-kappa B inhibitor is Silibinin.

Preferred methods include embodiments wherein said NF-kappa B inhibitor is Betulinic acid.

Preferred methods include embodiments wherein said NF-kappa B inhibitor is Ursolic acid.

Preferred methods include embodiments wherein said NF-kappa B inhibitor is Monochloramine.

Preferred methods include embodiments wherein said NF-kappa B inhibitor is glycine chloramine.

Preferred methods include embodiments wherein said NF-kappa B inhibitor is Anethole.

Preferred methods include embodiments wherein said NF-kappa B inhibitor is Baoganning.

Preferred methods include embodiments wherein said NF-kappa B inhibitor is cyanidin 3-O-glucoside.

Preferred methods include embodiments wherein said NF-kappa B inhibitor is cyanidin 3-O-(2(G)-xylosylrutinoside.

Preferred methods include embodiments wherein said NF-kappa B inhibitor is cyanidin 3-O-rutinoside.

Preferred methods include embodiments wherein said NF-kappa B inhibitor is Buddlejasaponin IV.

Preferred methods include embodiments wherein said NF-kappa B inhibitor is Cacospongionolide B.

Preferred methods include embodiments wherein said NF-kappa B inhibitor is Calagualine.

Preferred methods include embodiments wherein said NF-kappa B inhibitor is Cardamonin.

Preferred methods include embodiments wherein said NF-kappa B inhibitor is Cycloepoxydon.

Preferred methods include embodiments wherein said NF-kappa B inhibitor is 1-hydroxy-2-hydroxymethyl-3-pent-1-enylbenzene.

Preferred methods include embodiments wherein said NF-kappa B inhibitor is Decursin.

Preferred methods include embodiments wherein said NF-kappa B inhibitor is Dexanabinol.

Preferred methods include embodiments wherein said NF-kappa B inhibitor is Digitoxin.

Preferred methods include embodiments wherein said NF-kappa B inhibitor is Diterpenes.

Preferred methods include embodiments wherein said NF-kappa B inhibitor is Docosahexaenoic acid.

Preferred methods include embodiments wherein said NF-kappa B inhibitor is oxidized low density lipoprotein.

Preferred methods include embodiments wherein said NF-kappa B inhibitor is 4-Hydroxynonenal (HNE).

Preferred methods include embodiments wherein said NF-kappa B inhibitor is Flavopiridol.

Preferred methods include embodiments wherein said NF-kappa B inhibitor is [6]-gingerol.

Preferred methods include embodiments wherein said NF-kappa B inhibitor is casparol.

Preferred methods include embodiments wherein said NF-kappa B inhibitor is Glossogyne tenuifolia.

Preferred methods include embodiments wherein said NF-kappa B inhibitor is inositol hexakisphosphate.

Preferred methods include embodiments wherein said NF-kappa B inhibitor is Phytic acid.

Preferred methods include embodiments wherein said NF-kappa B inhibitor is Prostaglandin A1.

Preferred methods include embodiments wherein said NF-kappa B inhibitor is 20(S)-Protopanaxatriol.

Preferred methods include embodiments wherein said NF-kappa B inhibitor is Rengyolone.

Preferred methods include embodiments wherein said NF-kappa B inhibitor is Rottlerin.

Preferred methods include embodiments wherein said NF-kappa B inhibitor is Saikosaponin-d.

Preferred methods include embodiments wherein said NF-kappa B inhibitor is Cacospongionolide B.

Preferred methods include embodiments wherein said NF-kappa B inhibitor is xenon.

Preferred methods include embodiments wherein said NF-kappa B inhibitor is argon.

Preferred methods include embodiments wherein said NF-kappa B inhibitor is radon.

Preferred methods include embodiments wherein said NF-kappa B inhibitor is ozone.

Preferred methods include embodiments wherein said NF-kappa B inhibitor is fish oil.

Preferred methods include embodiments wherein said NF-kappa B inhibitor is carbon monoxide.

Preferred methods include embodiments wherein said health status is cardiovascular function.

Preferred methods include embodiments wherein said cardiovascular function is appropriate left ventricular ejection fraction as compared to healthy age matched individuals.

Preferred methods include embodiments wherein said cardiovascular function is appropriate QRS electrophysiology as compared to healthy age matched individuals.

Preferred methods include embodiments wherein said cardiovascular function is ejection fraction between 50-75%.

Preferred methods include embodiments wherein said cardiovascular function a heart rate of 63-93 beats per minute.

Preferred methods include embodiments wherein said cardiovascular function is a blood volume of 65-73 mL/kg.

Preferred methods include embodiments wherein said cardiovascular function is a mean arterial pressure of 63-93 mmHg.

Preferred methods include embodiments wherein said cardiovascular function is cardiac output of 5.9-6.5 L/min.

Preferred methods include embodiments wherein said cardiovascular function is stroke volume of 98-102) mL.

Preferred methods include embodiments wherein said cardiovascular function is 60-80 beats per minute.

Preferred methods include embodiments wherein said cardiovascular function is P-wave duration of 80-130 ms.

Preferred methods include embodiments wherein said cardiovascular function is QRS complex duration of 80 ms.

Preferred methods include embodiments wherein said cardiovascular function is a PR interval of 119 to 210 ms.

Preferred methods include embodiments wherein said cardiovascular function is a QT interval of 311 to 440 ms.

Preferred methods include embodiments wherein said cardiovascular function is a T wave axis of −8 to 740.

Preferred methods include embodiments wherein said cardiovascular function is a Sokolow-Lyon index 2.13 to 6.21 mV.

Preferred methods include embodiments wherein said cardiovascular function is a Cornell index 0.17 to 6.24 mV.

Preferred embodiments are drawn to methods of predicting propensity to develop a degenerative condition, comprising of: a) obtaining an aliquot of biological fluid; b) assessing inflammation associated stimuli found in said biological fluid; c) assessing regeneration associated stimuli in said biological fluid; and d) predicting inflammatory versus regenerative status of said subject from which said biological fluid was extracted.

Preferred methods include embodiments wherein said degenerative condition is selected from a group of conditions including: a) heart failure, b) liver failure; c) neurological deterioration; d) renal failure; e) ocular dysfunction; f) gastrointestinal dysfunction; g) hematopoietic dysfunction; h) dermatological abnormalities; i) urological abnormalities; j) skeletal abnormalities; k) respiratory abnormalities; 1) endocrine abnormalities; j) cardiovascular abnormalities; and k) reproductive abnormalities.

Preferred methods include embodiments wherein said abnormalities are as a result of natural aging.

Preferred methods include embodiments wherein said abnormalities are as a result of a viral infection.

Preferred methods include embodiments wherein said abnormalities are as a result of a bacterial infection.

Preferred methods include embodiments wherein said abnormalities are as a result of a fungal infection.

Preferred methods include embodiments wherein said abnormalities are as a result of a parasitic infection.

Preferred methods include embodiments wherein said abnormalities are as a result of an injury.

Preferred methods include embodiments wherein said abnormalities are as a result of radiation exposure.

Preferred methods include embodiments wherein said abnormalities are as a result of a cancer therapy.

Preferred methods include embodiments wherein said cancer therapies are selected from a group comprising of: methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, STI-571 or Gleevec.™. (imatinib mesylate), herbimycin A, genistein, erbstatin, and lavendustin A. taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostinsherbimycin A, genistein, erbstatin, and lavendustin ABCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, the anti-cancer agent can be, but is not limited to, a drug listed: Alkylating agents Nitrogen mustards: Cyclophosphamide Ifosfamide Trofosfamide Chlorambucil Nitrosoureas: Carmustine (BCNU) Lomustine (CCNU) Alkylsulphonates: Busulfan Treosulfan Triazenes: Dacarbazine Platinum containing Cisplatin compounds: Carboplatin Aroplatin Oxaliplatin Plant Alkaloids Vinca alkaloids: Vincristine Vinblastine Vindesine Vinorelbine Taxoids: Paclitaxel Docetaxel DNA Topoisomerase Inhibitors Epipodophyllins: Etoposide Teniposide Topotecan 9-aminocamptothecin Camptothecin Crisnatol mitomycins: Mitomycin C Anti-metabolites Anti-folates: DHFR inhibitors: Methotrexate Trimetrexate IMP dehydrogenase Mycophenolic acid Inhibitors: Tiazofurin Ribavirin EICAR Ribonuclotide reductase Hydroxyurea Inhibitors: Deferoxamine Pyrimidine analogs: Uracil analogs: 5-Fluorouracil Floxuridine Doxifluridine Ratitrexed Cytosine analogs: Cytarabine (ara C) Cytosine arabinoside Fludarabine Purine analogs: Mercaptopurine Thioguanine DNA Antimetabolites: 3-HP 2′-deoxy-5-fluorouridine 5-HP alpha-TGDR aphidicolin glycinate ara-C 5-aza-2′-deoxycytidine beta-TGDR cyclocytidine guanazole inosine glycodialdehyde macebecin II pyrazoloimidazole Hormonal therapies: Receptor antagonists: Anti-estrogen: Tamoxifen Raloxifene Megestrol LHRH agonists: Goserelin Leuprolide acetate Anti-androgens: Flutamide Bicalutamide Retinoids/Deltoids Cis-retinoic acid Vitamin A derivative: All-trans retinoic acid (ATRA-IV) Vitamin D3 analogs: EB 1089 CB 1093 KH 1060 Photodynamic therapies: Vertoporfin (BPD-MA) Phthalocyanine Photosensitizer Pc4 Demethoxy-hypocrellin A (2BA-2-DMHA) Cytokines: Interferon-.alpha. Interferon-.gamma. Tumor necrosis factor Angiogenesis Inhibitors: Angiostatin (plasminogen fragment) antiangiogenic antithrombin III Angiozyme ABT-627 Bay 12-9566 Benefin Bevacizumab BMS-275291 cartilage-derived inhibitor (CDI) CAI CD59 complement fragment CEP-7055 Col 3 Combretastatin A-4 Endostatin (collagen XVIII fragment) Fibronectin fragment Gro-beta Halofuginone Heparinases Heparin hexasaccharide fragment HMV833 Human chorionic gonadotropin (hCG) IM-862 Interferon alpha/beta/gamma Interferon inducible protein (IP-10) Interleukin-12 Kringle 5 (plasminogen fragment) Marimastat Metalloproteinase inhibitors (TIMPs) 2-Methoxyestradiol MMI 270 (CGS 27023A) MoAb IMC-1C11 Neovastat NM-3 Panzem PI-88 Placental ribonuclease inhibitor Plasminogen activator inhibitor Platelet factor-4 (PF4) Prinomastat Prolactin 16 kD fragment Proliferin-related protein (PRP) PTK 787/ZK 222594 Retinoids Solimastat Squalamine SS 3304 SU 5416 SU6668 SU11248 Tetrahydrocortisol-S tetrathiomolybdate thalidomide Thrombospondin-1 (TSP-1) TNP-470 Transforming growth factor-beta (TGF-b) Vasculostatin Vasostatin (calreticulin fragment) ZD6126 ZD 6474 farnesyl transferase inhibitors (FTI) bisphosphonates Antimitotic agents: allocolchicine Halichondrin B colchicine colchicine derivative dolstatin 10 maytansine rhizoxin thiocolchicine trityl cysteine Others: Isoprenylation inhibitors: Dopaminergic neurotoxins: 1-methyl-4-phenylpyridinium ion Cell cycle inhibitors: Staurosporine Actinomycins: Actinomycin D Dactinomycin Bleomycins: Bleomycin A2 Bleomycin B2 Peplomycin Anthracyclines: Daunorubicin Doxorubicin (adriamycin) Idarubicin Epirubicin Pirarubicin Zorubicin Mitoxantrone MDR inhibitors: Verapamil Ca.sup.2+ATPase inhibitors: Thapsigargin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Preferred methods include embodiments wherein said biological fluid is tear drops.

Preferred methods include embodiments wherein said biological fluid is blood.

Preferred methods include embodiments wherein said biological fluid is plasma.

Preferred methods include embodiments wherein said biological fluid is sweat.

Preferred methods include embodiments wherein said biological fluid is urine.

Preferred methods include embodiments wherein said biological fluid is serum.

Preferred methods include embodiments wherein said biological fluid is cerebral spinal fluid.

Preferred methods include embodiments wherein said inflammation associated stimuli is activated immune cells.

Preferred methods include embodiments wherein said activated immune cells are B cells.

Preferred methods include embodiments wherein said B cells are producing interferon gamma.

Preferred methods include embodiments wherein said B cells are producing TNF-alpha.

Preferred methods include embodiments wherein said B cells are producing interleukin-1.

Preferred methods include embodiments wherein said B cells are producing TNF-beta.

Preferred methods include embodiments wherein said B cells are producing interleukin-2.

Preferred methods include embodiments wherein said B cells are producing interleukin-6.

Preferred methods include embodiments wherein said B cells are producing interleukin-7.

Preferred methods include embodiments wherein said B cells are producing interleukin-8.

Preferred methods include embodiments wherein said B cells are producing interleukin-11.

Preferred methods include embodiments wherein said B cells are producing interleukin-12.

Preferred methods include embodiments wherein said B cells are producing interleukin-15.

Preferred methods include embodiments wherein said B cells are producing interleukin-17.

Preferred methods include embodiments wherein said B cells are producing interleukin-18.

Preferred methods include embodiments wherein said B cells are producing interleukin-33.

Preferred methods include embodiments wherein said B cells are deficient at producing interleukin-4.

Preferred methods include embodiments wherein said B cells are deficient at producing interleukin-10.

Preferred methods include embodiments wherein said B cells are deficient at producing interleukin-13.

Preferred methods include embodiments wherein said B cells are deficient at producing interleukin-20.

Preferred methods include embodiments wherein said B cells are deficient at producing TGF-beta.

Preferred methods include embodiments wherein said B cells are deficient at producing soluble HLA-G.

### DETAILED DESCRIPTION OF THE INVENTION

We describe through the invention the utilization of purified populations of mammalian endothelial stem cells as a means of predicting health and preventing deterioration of health by possessing knowledge which allows for alternation in lifestyle before adverse health events materialize. For the purpose of describing the invention in this specification, a stem cell means any immature cell that can develop into a mature cell of more than one type. The stem cell may be pluripotent (which express, amongst other markers, OCT-4, NANOG and SOX2), bipotent (which express, amongst others, VEGF-receptor), or monopotent (which express, amongst other markers IL-3 receptor). Monopotent stem cells are also referred to as progenitor cells. Through the quantification of such stem cells and/or progenitor cells, it is possible to understand and/or predict future health status of an individual. It is known in the art that pluripotent stem cells are capable of developing into more than two types of mature cells, such as endothelial cells, hematopoietic cells, and at least one other type of cells. Bipotent stem cells are capable of developing into two types of mature cells, such as endothelial cells and hematopoietic cells. Progenitor cells are capable of developing into one type of mature cells, such as endothelial cells or hematopoietic cells. Pluripotent stem cells, bipotent stem cells, and progenitor cells are capable of developing into mature cells either directly, or indirectly through one or more intermediate stem or progenitor cells.

In one embodiment quantification of pluripotent stem cells is provided in the circulation of a patient. Quantification can be performed by assessment of markers, and/or biological properties. It is known that an endothelial stem cell is a stem cell that is capable of maturing at least into mature endothelial cells. The endothelial stem cell may be pluripotent, bipotent, or monopotent. Monopotent endothelial stem cells are also referred to as endothelial progenitor cells.

In some embodiments, quantification is made of pluripotent endothelial stem cells are capable of developing into mature endothelial cells and at least two other types of cells, such as hematopoietic cells. Bipotent endothelial stem cells are capable of developing into mature endothelial cells and one other type of cells, such as hematopoietic cells. Monopotent endothelial cells, i.e. endothelial progenitor cells, are capable of developing into mature endothelial cells. The invention covers, intra alia, the concentration and quantification of blood forming stem cell such as hematopoietic stem cells. A hematopoietic stem cell is a stem cell that is capable of maturing at least into mature hematopoietic cells. The hematopoietic stem cell may be pluripotent, bipotent, or monopotent. Monopotent hematopoietic stem cells are also referred to as hematopoietic progenitor cells. Pluripotent hematopoietic stem cells are capable of developing into mature hematopoietic cells and at least two other types of cells, such as endothelial cells. Bipotent hematopoietic stem cells are capable of developing into mature hematopoietic cells and one other type of cells, such as endothelial cells. Monopotent hematopoietic stem cells, i.e. hematopoietic progenitor cells, are capable of developing into mature hematopoietic cells. Accordingly to the above definitions, the term pluripotent stem cell always includes bipotent stem cells and progenitor cells. The term bipotent stem cell always includes progenitor cells. For example, stem cells include, but are not limited to, hemangioblasts and angioblasts.

For the practice of the invention, endothelial stem cells are characterized by highly expressed surface antigens. Such antigens include, for example, one or more vascular endothelial growth factor receptors (VEGFR). Examples of VEGFRs include FLK-1 and FLT-1. The FLK-1 receptor is also known by other names, such as VEGFR-2. Human FLK-1 is sometimes referred to in the literature and herein as KDR. It is known that at least some endothelial stem cells also express the CD34+ marker. The endothelial stem cells may be-further characterized by the absence or significantly lower expression levels of certain markers characteristic of mature cells. Such markers include CD1, CD3, CD8, CD10, CD13, CD14, CD15, CD19, CD20, CD33, and CD41A. Cells lacking these markers will be referred to as Lin-. Most, if not all, of the endothelial stem cells express high levels of FLK-1. The CD34 marker is characteristic of stem cells, such as angioblasts and hematopoietic stem cells. Approximately 0.5-10% of CD34+ cells are also FLK-1+. For example, approximately 1% of bone marrow cells are CD34+. Of these, approximately 1% are FLK-1+.

In one embodiment, the method relates to a method of isolating populations of endothelial stem cells and quantifying their biological activity. The population of endothelial stem cells is purified. By purified is meant that the population is significantly enriched in endothelial stem cells from the crude population of cells from which the endothelial stem cells are isolated. Assessment of biological activity is performed by quantifying levels of cytokines produced at a basal level and/or cytokines produced with the cells are activity. Cytokines assessed include FGF-1, FGF-2, FGF-5, VEGF, KGF, HGF-1, PDGF-BB, angiopoietin, and growth hormone. Stimulators of stem cells include danger signals such as the toll receptor family. The purification procedure should lead at least to a five fold increase, preferably at least a ten fold increase, more preferably at least a fifteen fold increase, most preferably at least a twenty fold increase, and optimally at least a twenty-five fold increase in endothelial stem cells over the total population. The purified population of endothelial stem cells should include at least 15%, preferably at least 20%, more preferably at least 25%, most preferably at least 35%, and optimally at least 50% of endothelial stem cells. The molecule used to separate endothelial stem cells from the contaminating cells can be any molecule that binds specifically to the antigen that characterizes the endothelial stem cell. The molecule can be, for example, a monoclonal antibody, a fragment of a monoclonal antibody, or, in the case of an antigen that is a receptor, the ligand of that receptor. For example, in the case of a VEGF receptor, such as FLK-1, the ligand is VEGF. The number of antigens characteristic of endothelial stem cells found on the surface of such cells is sufficient to isolate purified populations of such cells. For example, the number of antigens found on the surface of endothelial stem cells should be at least approximately 5,000, preferably at least approximately 10,000, more preferably at least approximately 25,000, and most preferably at least approximately 50,000. There is no limit as to the number of antigens contained on the surface of the cells. For example, the cells may contain approximately 150,000, 250,000, 500,000, 1,000,000, or even more antigens on the surface. The endothelial progenitor cells used in the methods described herein can either be autologous cells, modified ex vivo to contain a functional form of the gene encoding for a deficient protein; or they can be heterologous cells, isolated from a subject with a functional form of the deficient protein if needed. Autologous EPCs can be obtained from a patient's peripheral blood, bone marrow or umbilical cord blood. EPCs can be isolated using antibodies that specifically recognize EPC antigens on immature human hematopoietic progenitor cells (HSCs). For example, antibodies against CD34 and Flk-1 can be used to isolate EPCs from a population of HSCs. Methods for obtaining HSCs are disclosed in, e.g., U.S. Pat. No. 5,199,942. Furthermore, EPCs can also be isolated directly from the peripheral blood or umbilical cord blood, and expanded ex vivo. See, for example, Kalka et al., PNAS, Vol. 97(7), pp. 3422-3427, Mar. 28, 2000, and Boyer et al., J Vase Surg, 31(1 Pt 1), pp.181-189, January 2000. Isolation of heterologous EPCs can be performed in the same manner described for autologous cells, with a distinction that the heterologous cells are isolated from a healthy donor rather than from a patient. The healthy donor herein refers to a subject who expresses a functional form of the protein that is deficient in the subject to be treated, and who has no diseases that could be transmitted by administration of his/her cells to the afflicted or predisposed patient, such as, for instance, Hepatitis B. In one embodiment, it is desirable that the donor is MHC-matched to the patient who is being treated with the donor's EPCs. MHC-matching can be performed using a mixed lymphocyte reaction (MLR) or a cytotoxic T-cell assay (CTL assay). Following isolation, EPCs, either autologous or heterologous, are modified in culture to express the functional version of a gene coding for a defective protein. It should be noted, however, that heterologous EPCs do not need to be modified if the protein that is deficient in a patient to be treated is expressed and secreted by endothelial cells of the healthy subject, whose EPCs are used as a source of heterologous cells. For example, treating a patient with Hemophilia A with heterologous cells does not require genetically-modifying said heterologous cells since they express and secrete Factor VIII, the protein that is deficient in patients with Hemophilia A. However, even in such cases, where heterologous endothelial cells secrete a protein that is deficient in a patient with the congenital protein deficiency, it may be desirable to transfect heterologous EPCs with a gene encoding a functional form of the patient's deficient protein in order to increase the expression and secretion of said protein upon EPC engraftment.

The invention is based on the discovery an endothelial progenitor cell. The progenitor cell is multipotent. The progenitor cell is capable of differentiating, e.g., in-vivo or in-vitro, into a endothelial cell or a smooth muscle cell. Accordingly, the invention features a endothelial progenitor cell culture, e.g., an in-vitro culture. The culture is an adhesion culture. Alternatively, the cells in the culture are in suspension. The cell is derived from hematopoietic tissue, such as blood, e.g. peripheral blood or bone marrow. The tissue is from a mammal such as human, a primate, mouse, rat, dog, cat, cow, horse, pig. The cell is immunoreactive for VEGFR-2 or Tie-2 and CD45 and non-immunoreactive with CD144. Optionally the VEGFR.sup.+ cell is also immunoreactive with CD14. The cells proliferate in vitro. The cells are capable of doubling 2, 3, 4, 5, 6, 7, 8, 9 10, 11, 12, 15, 20, 25 or more times and maintain there ability to differentiate into endothelial cells or smooth muscle cells. The invention further features a method of identifying the progenitor cell or the progenitor cell progeny in subject by providing VEGFR-2.sup.+ or Tie-2.sup.+, CD45.sup.+ and CD144.sup.- progenitor cells or VEGFR-2.sup.+CD14.sup.+, CD45.sup.+ and CD144.sup.- progenitor cells and transplanting the cells into the subject. Transplantation confers a clinical benefit, e.g. alleviating one or more symptoms of the particular vascular disorder. Vascular disorders are diagnosed by a physician using methods know in the art. The ability to quantify ability of regenerative cells and/or EPC to function properly can be assessed by activity in vitro in repairing endothelial monolayers or in vivo. An injured blood vessel is repopulated by administering to a subject a composition containing VEGFR-2.sup.+ or Tie-2.sup.+, CD45.sup.+ and CD144.sup.- progenitor cells or a composition containing VEGFR-2.sup.+CD14.sup.+, CD45.sup.+ and CD144.sup.- progenitor cells. The composition is administered directly to the blood vessel (e.g., vein or artery), or arterial bed. Alternatively the composition is administered systemically. In some embodiments the functional activity is assessed by ability to endothelized an injured vessel by contacting a vascular grafts with a composition containing VEGFR-2.sup.+ or Tie-2.sup.+, CD45.sup.+ and CD144.sup.-progenitor cells or a composition containing VEGFR-2.sup.+CD14.sup.+, CD45.sup.+ and CD144.sup.- progenitor cells. The vascular graft is a vein. Alternatively the vascular graft is a artery. The graft is an artificial vascular graft, an allograft, or an isograft. The graft is contacted in vitro, in vivo, or ex vivo. The vascular graft is contacted prior to implantation in a subject. Alternatively, the graft is contacted with the composition after implantation in a subject. By repopulating a blood vessel or endothelizing an vascular graft is meant that the blood vessel or graft has an increased amount of endothelial cells compared to blood vessel of graft that has not been treated with the progenitor cells. For example, the treated blood vessel or graft has an increased vessel diameter and media thickness as compared to an untreated blood vessel or graft.

One of the cell types being quantified as a measure of predicting health is the VEGFR-2.sup.+ or Tie-2.sup.+, CD45.sup.+ and CD144.sup.- progenitor cells, which maintain the capacity to differentiated in vivo to endothelial cells or smooth muscle cells. Whereas the VEGFR-2.sup.+CD14.sup.+, CD45.sup.+ and CD144.sup.31 progenitor cells maintain the capacity to differentiated in vivo to endothelial cells. The subject is for example a mammal such as human, a primate, mouse, rat, dog, cat, cow, horse, pig. The subject is suffering from a vascular disorder or vascular tissue damage. For example, the subject is suffering from Peripheral Artery Disease, Aneurysm, Renal (Kidney) Artery Disease, Raynaud's Phenomenon, Buerger's Disease, Peripheral Venous Disease, Varicose Veins, Blood Clots In the Veins. Transplantation and/or repopulation confers a clinical benefit, e.g., alleviating one or more symptoms of the particular vascular disorder. Vascular disorders are diagnosed by a physician using methods know in the art. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Tissue injury and hypoxia are known to generate chemoattractants that potentially are responsible for mobilization of EPC. Reduction in oxygen tension occurs as a result of numerous injuries including stroke, infarction, or contusion. Oxygen tension is biologically detected by the transcription factor HIF-1 alpha, which upon derepression undergoes nuclear translocation. This event causes upregulated expression of a plethora of angiogenesis promoting cytokines and chemoattractants [43], such as stromal derived factor (SDF)-1 and VEGF [44, 45]. On the other hand, tissue necrosis causes release of “danger signals” such as HMBG1, a nuclear factor that has direct chemoattractant activity on mesoangioblasts, a type of EPC [46, 47]. It has been demonstrated that this systemic release of chemoattractant cytokines after vascular injury or infarct is associated with mobilization of endogenous bone marrow cells and EPC [48].

Myocardial infarction has been widely studied in the area of regenerative medicine in which cellular and molecular aspects of host response post-injury are relatively well defined. EPC mobilization after acute ischemia has been demonstrated in several cardiac infarct studies. This was first reported by Shintani et al who observed increased numbers of CD34 positive cells in 16 post infarct patients on day 7 as compared to controls. The rise in CD34 cells correlated with ability to differentiate into cells morphologically resembling endothelium and expressing endothelial markers KDR and CD31. Supporting the concept that response to injury stimulates EPC mobilization, a rise in systemic VEGF levels was correlated with increased EPC numbers [45]. A subsequent study demonstrated a similar rise in circulating EPC post infarct. Blood was drawn from 56 patients having a recent infarct (<12 hours), 39 patients with stable angina, and 20 healthy controls. Elevated levels of cells expressing CD34/CXCR4+ and CD34/CD117+ and c-met+ were observed only in the infarct patients which were highest at the first blood draw. In this study the mobilized cells not only expressed endothelial markers, but also myocytic and cardiac genes [49]. The increase in circulating EPC at early timepoints post infarction has been observed by other groups, and correlated with elevations in systemic VEGF and SDF-1 [50, 51].

In the case of cerebral infarction studies support the concept that not only are EPC mobilized in response to ischemia, but also that the extent of mobilization may be associated with recovery. In a trial of 48 patients suffering primary ischemic stroke, mobilization of EPC was observed in the first week in comparison to control patients. EPC were defined as cells capable of producing endothelial colony forming units. A correlation between improved outcome at 3 months and extend of EPC mobilization was observed based on the NIHSS and Rankin score [52]. In a similar study, Dunac et al reported on circulating CD34 levels of 25 patients with acute stroke for 14 days. A correlation between improvement on the Rankin scale and increased circulating CD34 cells was reported [53]. Noteworthy was that the level of CD34 mobilization was similar to that observed in patients treated with the mobilize G-CSF. In a larger study, Yip et al examined EPC levels in 138 consecutive patients with acute stroke and compared them to 20 healthy volunteers and in 40 at-risk control subjects [54]. Three EPC phenotypes were assessed by flow cytometry at 48 hours after stroke: a) CD31/CD34, b) CD62E/CD34, and c) KDR/CD34. Diminished levels of all three EPC subsets in circulation was predictive of severe neurological impairment NIHSS>/=12, while suppressed levels of circulating CD31/34 cells was correlated with combined major adverse clinical outcomes as defined by recurrent stroke, any cause of death, or NIHSS>/=12. Increased levels of the KDR/CD34 phenotype cells was strongly associated with NIHSS> or =4 on day 21. Although these studies do not directly demonstrate a therapeutic effect of the mobilized EPC, animal studies in the middle cerebral artery ligation stroke model have demonstrated positive effects subsequent to EPC administration [55, 56], an effect which appears to be at least partially dependent on VEGF production from the EPC [57].

Another ischemia-associated tissue insult is acute respiratory distress syndrome (ARDS), in which respiratory failure often occurs as a result of disruption of the alveolar-capillary membrane, which causes accumulation of proteinaceous pulmonary edema fluid and lack of oxygen uptake ability [58]. In this condition there has been some speculation that circulating EPC may be capable of restoring injured lung endothelium. For example, it is known that significant chimerism (37-42%) of pulmonary endothelial cells occurs in female recipients of male bone marrow transplants [59]. Furthermore, in patients with pneumonia infection there is a correlation between infection and circulating EPC, with higher numbers of EPC being indicative of reduced fibrosis [60]. The possibility that EPC are mobilized during ARDS and may be associated with benefit was examined in a study of 45 patients with acute lung injury in which a correlation between patients having higher number of cells capable of forming endothelial colonies in vitro and survival was made. Specifically, the patients with a colony count of > or =35 had a mortality of approximately 30%, compared to patients with less than 35 colonies, which had a mortality of 61%. The correlation was significant after multivariable analysis correcting for age, sex, and severity of illness [61]. From an interventional perspective, transplantation of EPC into a rabbit model of acute lung injury resulted in reduction of leukocytic infiltrates and preservation of pulmonary cellular integrity [62].

Sepsis is a major cause of ARDS and is associated with acute systemic inflammation and vascular damage. Septic patients have elevated levels of injury associated signals and EPC mobilizers such as HMGB1 [63], SDF-1 [64], and VEGF [65]. Significant pathology of sepsis is associated with vascular leak and disseminated intravascular coagulation [66]. The importance of the vasculature in sepsis can perhaps be supported by the finding that the only drug to have an impact on survival, Activated Protein C, acts primarily through endothelial protection [67]. Septic patients are known to have increased circulating EPC as compared to controls. Becchi et al observed a correlation between VEGF and SDF-1 levels with a 4-fold rise in circulating EPC in septic patients as compared to healthy controls [64]. A correlation between EPC levels and survival after sepsis was reported in a study of 32 septic patients, 15 ICU patients, and 15 controls. Of the 8 patients who succumbed to sepsis by 28 days, as compared to 24 survivors, a significantly reduced EPC number in non-survivors was reported [68].

It appears that in conditions of acute injury, elevation of EPC in circulation occurs. Although studies in stroke [52-54], ARDS [61], and sepsis [68] seem to correlate outcome with extend of mobilization, work remains to be performed in assessing whether it is the EPC component that is responsible for benefits or other confounding variables. Taking into account the possibility that EPC may act as an endogenous repair mechanism, we will discuss data in chronic degenerative conditions in which circulating EPC appear to be suppressed.

The mixture of cells obtained are exposed to a molecule that binds specifically to the antigen marker characteristic of endothelial stem cells. The molecule is preferably an antibody or a fragment of an antibody. A convenient antigen marker is a VEGF receptor, more specifically a FLK-1 receptor. The cells that express the antigen marker bind to the molecule. The molecule distinguishes the bound cells from unbound cells, permitting separation and isolation. If the bound cells do not internalize the molecule, the molecule may be separated from the cell by methods known in the art. For example, antibodies may be separated from cells with a protease such as chymotrypsin. The molecule used for isolating the purified populations of endothelial stem cells is advantageously conjugated with labels that expedite identification and separation. Examples of such labels include magnetic beads, biotin, which may be removed by avidin or streptavidin, fluorochromes, which may be used in connection with a fluorescence-activated cell sorter, and the like. Cells that are bound to the molecule are removed from the cell suspension by physically separating the solid support from the cell suspension. For example, the unbound cells may be eluted or washed away with physiologic buffer after allowing sufficient time for the solid support to bind the endothelial stem cells. The bound cells are separated from the solid phase by any appropriate method, depending mainly upon the nature of the solid phase and the molecule. For example, bound cells can be eluted from a plastic petri dish by vigorous agitation. Alternatively, bound cells can be eluted by enzymatically “nicking” or digesting an enzyme-sensitive “spacer” sequence between the solid phase and an antibody. Suitable spacer sequences bound to agarose beads are commercially available from, for example, Pharmacia. The eluted, enriched fraction of cells may then be washed with a buffer by centrifugation and preserved in a viable state at low temperatures for later use according to conventional technology. The cells may also be used immediately, for example by being infused intravenously into a recipient. In a preferred embodiment, a labeled molecule is bound to the endothelial stem cells, and the labeled cells are separated by a mechanical cell sorter that detects the presence of the label. The preferred mechanical cell sorter is a florescence activated cell sorter (FACS). FACS machines are commercially available. Generally, the following FACS protocol is suitable for this procedure: A Coulter Epics Eliter sorter is sterilized by running 70% ethanol through the systems. The lines are flushed with sterile distilled water. Cells are incubated with a primary antibody diluted in Hank's balanced salt solution supplemented with 1% bovine serum albumin (HB) for 60 minutes on ice. The cells are washed with HB and incubated with a secondary antibody labeled with fluorescein isothiocyanate (FITC) for 30 minutes on ice. The secondary label binds to the primary antibody. The sorting parameters, such as baseline fluorescence, are determined with an irrelevant primary antibody. The final cell concentration is usually set at one million cells per ml. While the cells are being labeled, a sort matrix is determined using fluorescent beads as a means of aligning the instrument. Once the appropriate parameters are determined, the cells are sorted and collected in sterile tubes containing medium supplemented with fetal bovine serum and antibiotics, usually penicillin, streptomycin and/or gentamicin. After sorting, the cells are re-analyzed on the FACS to determine the purity of the sort. In another embodiment, the invention is directed to purified populations of stem cells that express a VEGF receptor, such as, for example, the FLK-1 receptor. This embodiment further includes isolation of purified populations of such cells. The VEGFR+ stem cells include, for example, endothelial stem cells or hematopoietic stem cells. The source of cells from which the stem cells are obtained include both pre-natal and post-natal sources. Post-natal sources are preferred. The definitions and methods in this specification used in conjunction with purified populations of endothelial stem cells apply as well to the purified populations of stem cells that express a VEGF receptor.

There is need for angiogenesis and tissue remodeling in the context of various chronic inflammatory conditions. However in many situations it is the aberrant reparative processes that actually contribute to the pathology of disease. Examples of this include: the process of neointimal hyperplasia and subsequent plaque formation in response to injury to the vascular wall [69], the process of hepatic fibrosis as opposed to functional regeneration [70], or the post-infarct pathological remodeling of the myocardium which results in progressive heart failure [71]. In all of these situations it appears that not only the lack of regenerative cells, but also the lack of EPC is present. Conceptually, the need for reparative cells to heal the ongoing damage may have been so overwhelming that it leads to exhaustion of EPC numbers and eventual reduction in protective effect. Supporting this concept are observations of lower number of circulating EPC in inflammatory diseases, which may be the result of exhaustion. Additionally, the reduced telomeric length of EPC in patients with coronary artery disease [72], as well as reduction of telomere length in the EPC precursors that are found in the bone marrow [73, 74] suggests that exhaustion in response to long-term demand may be occurring. If the reparatory demands of the injury indeed lead to depletion of EPC progenitors, then administration of progenitors should have therapeutic effects.

Several experiments have shown that administration of EPC have beneficial effects in the disease process. For example, EPC administration has been shown to: decrease balloon injury induced neointimal hyperplasia [75], b) suppress carbon tetrachloride induced hepatic fibrosis [76, 77], and inhibit post cardiac infarct remodeling [78]. One caveat of these studies is that definition of EPC was variable, or in some cases a confounding effect of coadministered cells with regenerative potential may be present. However, overall, there does appear to be an indication that EPC play a beneficial role in supporting tissue regeneration. As discussed below, many degenerative conditions, including healthy aging, are associated with a low-grade inflammation. There appears to be a causative link between this inflammation and reduction in EPC function.

Inflammatory conditions present with features, which although not the rule, appear to have commonalities. For example, increases in inflammatory markers such as C-reactive protein (CRP), erythrocyte sedimentation rate, and cytokines such as TNF-alpha and IL-18 have been described in diverse conditions ranging from organ degenerative conditions such as heart failure [79, 80], kidney failure [81, 82], and liver failure [83, 84] to autoimmune conditions such as rheumatoid arthritis [85] and Crohn's Disease [86], to healthy aging [87, 88]. Other markers of inflammation include products of immune cells such as neopterin, a metabolite that increases systemically with healthy aging [89], and its concentration positively correlates with cognitive deterioration in various age-related conditions such as Alzheimer's [90]. Neopterin is largely secreted by macrophages, which also produce inflammatory mediators such as TNF-alpha, IL-1, and IL-6, all of which are associated with chronic inflammation of aging [91]. Interestingly, these cytokines are known to upregulate CRP, which also is associated with aging [92]. While there is no direct evidence that inflammatory markers actively cause shorted lifespan in humans, strong indirect evidence of their detrimental activities exists. For example, direct injection of recombinant CRP in healthy volunteers induces atherothrombotic endothelial changes, similar to those observed in aging [93]. In vitro administration of CRP to endothelial cells decreases responsiveness to vasoactive factors, resembling the human age-associated condition of endothelial hyporesponsiveness [94].

Another important inflammatory mediator found elevated in numerous degenerative conditions is the cytokine TNF-alpha. Made by numerous cells, but primarily macrophages, TNF-alpha is known to inhibit proliferation of repair cells in the body, such as oligodendrocytes in the brain [95], and suppress activity of endogenous stem cell pools [96, 97]. TNF-alpha decreases EPC viability, an effect that can be overcome, at least in part by antioxidant treatment [98]. Administration of TNF-alpha blocking agents has been demonstrated to restore both circulating EPC, as well as endothelial function in patients with inflammatory diseases such as rheumatoid arthritis [18, 99, 100],

It appears that numerous degenerative conditions are associated with production of inflammatory mediators, which directly suppress EPC production or activity. This may be one of the reasons for findings of reduced EPC and FMD indices in patients with diverse inflammatory conditions. In addition to the direct effects, the increased demand for de novo EPC production in inflammatory conditions would theoretically lead to exhaustion of EPC precursors cells by virtue of telomere shortening

On average somatic cells can divide approximately 50 times, after which they undergo senescence, die or become cancerous. This limited proliferative ability is dependent on the telomere shortening problem. Every time cells divide the ends of the chromosomes called “telomeres” (complexes of tandem TTAGGGG repeats of DNA and proteins), are not completely replicated, thus they progressively get shorter [101]. Once telomeres reach a critical limit p53, p21, and p16 pathways are activated as a DNA damage response reaction instructing the cell to exit cell cycling. Associated with the process of senescence, the cells start expressing inflammatory cytokines such as IL-1 [102, 103], upregulation of adhesion molecules that attract inflammatory cells such as monocytes [104, 105], and morphologically take a flattened, elongated appearance. Physiologically, the process of cellular senescence caused in response to telomere shortening is believed to be a type of protective mechanism that cells have to prevented carcinogenesis [106]. At a whole organism level the association between telomere length and age has been made [107], as well, disorders of premature aging such as ataxia telangiectasia are characterized by accelerated telomere shortening [108].

The importance of this limited proliferative ability becomes apparent in our discussion of EPC. In general there is a need for continual endothelial cell replacement from EPC. Because the endothelial cells are exposed to enormous continual sheer stress of blood flow, mechanisms of repair and proliferation after injury need to exist. Theoretically, the more sheer stress on a particular artery, the more cell division would be required to compensate for cell loss. Indeed this appears to be the case. For example, telomeres are shorter in arteries associated with higher blood flow and sheer stress (like the iliac artery) as compared to arteries of lower stress such as the mammary artery [109]. The theory that senescence may be associated with atherosclerosis is supported since the iliac artery, which is associated with higher proliferation of endothelial cells and is also at a higher risk of atherosclerosis, thus prompting some investigators to propose atherosclerosis being associated with endothelial senescence [110, 111].

In an interesting intervention study Satoh et al examined 100 patients with coronary artery disease and 25 control patients. Telomere lengths were reduced in EPC of coronary artery disease patients as compared to controls. Lipid lowering therapy using agents such as atorvastatin has previously been shown to reduced oxidative stress and increase circulating EPC. Therapy with lipid lowering agents in this study resulted in preservation of telomeric length, presumably by decreasing the amount of de novo EPC produced, as well as oxidative stress leading to telomere erosion [112]. One important consideration when discussing telomere shortening of EPC is the difference between replicative senescence, which results from high need for differentiated endothelial cells, and stress induced senescence, in which inflammatory mediators can directly lead to telomere shortening. For example, smoking associated oxidative stress has been linked to stress induced senescence in clinical studies [113], whereas other studies have implicated inflammatory agents such as interferon gamma [114], TNF-alpha [115], and oxidative mediators as inducers of stress induced senescence [116].

Based on the above descriptions, it appears that in degenerative conditions, as well as in aging, an underlying inflammatory response occurs that is directly or indirectly associated with inhibition of circulating EPC activity. Directly, inflammation is known to suppress stem cell turnover and activity of EPC. Indirectly, inflammatory conditions place increased demands on the EPC progenitors due to overall increased need for EPC. Accordingly, an intervention strategy may be reduction in inflammatory states: this may be performed in a potent means by administration of agents such as TNF blockers [55], or more chronically by dietary supplements [117, 118], caloric restriction [119], exercise [120, 121], consuming blueberries [122], green tea [123], or statin therapy [124]. One example of a large scale intervention was the JUPITER trial of >17,000 healthy persons without hyperlipidemia but with elevated high-sensitivity C-reactive protein levels, Crestor significantly reduced the incidence of major cardiovascular incidents as well as lowering CRP levels [124]. Crestor has been shown to increase circulating EPC levels in vivo [125], in part through reduction of detrimental effects of asymmetric dimethylarginine on EPC [126]. Besides attempting to reduce inflammation, administration of EPC is another therapeutic possibility. The area of cardiac regeneration has been subject to most stem cell investigation besides hematopoietic reconstitution. Specifically, several double blind studies have been performed demonstrating overall increased cardiac function and reduction in pathological remodeling subsequent to administration of autologous bone marrow mononuclear cells [127-129]. Original thoughts regarding the use of bone marrow stem cells in infarcts revolved around studies showing “transdifferentiation” of various bone marrow derived cells into cells with myocardial features [130, 131]. While this concept is attractive, it has become very controversial in light of several studies demonstrating extremely minute levels of donor-derived cardiomyocytes, despite clinical improvement [132, 133]. An idea that has attracted interest is that bone marrow cells contain high numbers of EPC [134], so the therapeutic effect post infarct may not necessarily need to be solely based on regeneration via transdifferentiation, but via production of new blood vessels in the injured myocardium mediated by administered EPC in the bone marrow [135]. This view is supported by studies demonstrating that administration of EPC in other conditions of injury or fibrotic healing results in reduced tissue damage and organ functionality.

Instead of administering EPC another therapeutic possibility is to “reposition” them or simply to mobilize them from bone marrow sources. As previously discussed, myocardial and cerebral infarcts seem to cause a “natural mobilization”, which may be part of the endogenous response to injury. These observations led investigators to assess whether agents that mobilize EPC may be used therapeutically. Granulocyte colony stimulating factor (G-CSF) has been used clinically for mobilization of hematopoietic stem cells (HSC) for more than a decade during donor stem cell harvesting. Mechanistically G-CSF is believed to induce a MMP-dependent alteration of the SDF-1 gradient in the bone marrow [136, 137], as well as function through a complement-dependent remodeling of the bone marrow extracellular matrix [138, 139]. It was found that in addition to mobilizing HSC, G-CSF stimulates mobilization of EPC as well, through mechanisms that are believed to be related [35, 140]. Several studies have been performed in which G-CSF was administered subsequent to infarct. Although it is impossible to state whether the mobilization of HSC or EPC accounted for the beneficial effects, we will overview some of these studies.

The Front-Integrated Revascularization and Stem Cell Liberation in Evolving Acute Myocardial Infarction by Granulocyte Colony-Stimulating Factor (FIRSTLINE-AMI) trial evaluated 30 patients with ST-elevation myocardial infarction treated with control or G-CSF after successful revascularization [141]. Fifteen patients received 6 days of G-CSF at 10 μg/kg body weight, whereas the other 15 received standard care only. Four months after the infarct, the group that received G-CSF possessed a thicker myocardial wall at the area of infarct, as compared to controls. This was sustained over a year. Statistically significant improvements in ejection fraction, as well as inhibition of pathological remodeling was observed in comparison to controls. A larger subsequent study with 114 patients, 56 treated and 58 control demonstrated “no influence on infarct size, left ventricular function, or coronary restenosis” [142]. There may be a variety of reasons to explain the discrepancy between the trials. One most obvious one is that the mobilization was conducted immediately after the heart attack, whereas it may be more beneficial to time the mobilization with the timing of the chemotactic gradient released by the injured myocardium. This has been used to explain discrepancies between similar regenerative medicine trials [143]. Supporting this possibility is a study in which altered dosing was used for the successful improvement in angina [144]. Furthermore, a recent study last year demonstrated that in 41 patients with large anterior wall AMI an improvement in LVEF and diminished pathological remodeling was observed [145]. Thus while more studies are needed for definitive conclusions, it appears that there is an indication that post-infarct mobilization may have a therapeutic role. In the future, other clinically-applicable mobilizers may be evaluated. For example, growth hormone, which is used in “antiaging medicine” has been demonstrated to improve endothelial responsiveness in healthy volunteers [146], and patients with congestive heart failure [147], this appears to be mediated through mobilization of endothelial progenitor cells [148, 149].

One area of recent interest in the biomedical field has been functional foods and nutraceuticals. While it is known that alteration of diet may modulate FMD responses, to our knowledge, little work as been reported on dietary-supplements altering levels of circulating EPC. The nutritional supplement StemKine (Aidan Products, Chandler, AZ) contains: ellagic acid a polyphenol antioxidant found in numerous vegetables and fruits; vitamin D3 which has been shown to mildly increase circulating progenitor cells; beta 1,3 glucan (previous studies have reported administration of various beta glucans to elicit stem cell mobilization [150]), and a ferment of the bacterium, Lactobacillus fermentum. Lactobacillus fermentum is generally regarded as safe, and has been in the food supply for hundreds of years as a starter culture for the production of sour dough bread and provides for its characteristic sour flavor. Extract of green tea, extract of goji berries, and extract of the root of astragalus were added prior to the fermentation process. Green tea extracts and some components of goji berries are known to mildly stimulate progenitor cell release, and astragalosides and other molecules found in the root of astragalus are known antioxidants that can prevent cellular damage secondary to oxidation. Fermentation is known to increase the bioavailability of minerals, proteins, peptides, antioxidants, flavanols and other organic molecules. ImmKine, another Lactobacillus fermentum fermented product that includes beta 1,3, glucan has been safely distributed for 9 years without reported side effects.

We report here data from 6 healthy volunteers supplemented with StemKine (under and approved IRB protocol) for a period of 14 days (two capsules, am, two capsules pm, by mouth—700 mg per capsule). To our knowledge this is the first report of a combination of naturally occurring molecules from food products altering the levels of circulating EPCs in humans.

We report here data from 6 healthy volunteers supplemented with 4 capsules per day, each of 700 mg of StemKine for a period of 25 days. As seen in FIG. 1, an increase in cells expressing VEGFR2 and CD34 was observed, which was maintained for at least 14 days. These data suggest the feasibility of modulating circulating EPC levels using food supplements. Future studies integrating natural products together with regenerative medicine concepts may lead to formulation of novel treatment protocols applicable to age-associated degeneration.

Models for stem cell differentiation leading to endothelial and hematopoietic cells are of interest because of the clinical value of stem cells and their progeny. A hallmark of stem and progenitor cells is their ability to proliferate and give rise to functional progeny, and progenitor cells are identified by their clonogenic potential. Methods previously used do not guarantee that single endothelial cells have been isolated and characterized to identify the progenitors. One embodiment of the current invention, progenitor cells from the blood are extracted and ex vivo allowed to proliferate in order to identify and proactively predict health status of a subject that currently is healthy. For example, in one embodiment, blood is extracted and subjected to centrifugation with a density gradient in a manner to sufficiently collect mononuclear cells that are viable. Said collected mononuclear cells are cultured in a media that allows for proliferation of cells possessing regenerative potential. In some aspects of the invention, mononuclear cells are cultured in an endothelial cell supporting media. Such media are widely known in the art and include standard “backbone” media such as DMEM, EMEM, Alpha-MEM, RPMI and Iscove's Media. Various other stem cell/regenerative cell/endothelial progenitor cell media are available for use in the practice of the invention. For example, media may be supplement with cytokines such as bone morphogenic protein 4 (BMP4), basic fibroblast growth factor (bFGF), and vascular endothelial growth factor (VEGF) to generate conditions in which EPC or vascular progenitor cells can survive and be treated to assess activity and potency. In some aspects, the vascular progenitor cell is CD34+. In some aspects, the vascular progenitor also expresses at least one cell surface marker selected from CD31 (PECAM-1), c-Kit, and KDR. In some aspects, the vascular progenitor cell does not express CD 133. In some aspects, the culturing is carried out in the absence of feeder cells and/or Activin-A. In some aspects, the culturing is carried out in the absence of serum, that is, the culture solution (media) will lack serum. In some aspects, the solution includes about 5-100 ng/ml BMP4, e.g., about 10-80, 20-50, 20-40, 10-50, 20-30, or about 25 mg/ml BMP4. In some aspects, the solution includes about 5-100 ng/ml bFGF, e.g., about 10-70, 10-50, 15-40, 15-25, or about 20 mg/ml bFGF. In some aspects, the solution includes about 5-200 ng/ml VEGF, e.g., 10-100, 20-80, 30-60, 40-60, 45-55, or about 50 ng/ml VEGF. The methods of generating vascular progenitor cells can be carried out in less than 10 days of culture, but typically more than 2, 3, 4, 5, 6, or 7 days. In some aspects, the pluripotent stem cell is cultured for at least 4, 5, 6, 7, or 8 days, e.g., 6-9, 4-10, 5-10, 6-12, 7-12, 8-12, or 7-9 days. One of skill will understand that, during the culturing period, the initial pluripotent stem cell or population of pluripotent stem cells can divide and/or change character as the pluripotent stem cell gives rise to a vascular progenitor cell. Indeed, the method is typically carried out with a plurality of pluripotent stem cells to form a population of cells that includes, over time, increasing numbers of vascular progenitor cells. In some aspects, the population of cells comprises 30-80% vascular progenitor cells, e.g., 35, 40, 45, 50, 60, 65, 70, 75%, or higher percentage vascular progenitor cells. In some cases, the vascular progenitor cells are separated from the population of cells, e.g., for further differentiation. Such separation can be carried out using any method known in the art, e.g., based on cell surface marker expression, cell size, or cell morphology. Exemplary methods for cell separation include use of antibodies to cell surface markers (e.g., CD34, CD31, c-kit, and/or KDR for vascular progenitor cells) such as magnetic cell separation, sorting by flow cytometry, or chromatographic methods. For separation based on size, centrifugation, e.g., size exclusion centrifugation or density gradient centrifugation, can be used. See, e.g., Recktenwald (1998) Cell Separation Methods and Applications (Marcel Dekker ed.). In some aspects, the invention provides methods for differentiation of vascular progenitor cells into, e.g., endothelial cells (ECs), smooth muscle cells (SMCs), or cells of hematopoietic lineage, e.g., erythroid, granulocyte-macrophage, or macrophage cell types. Such methods can be practiced in combination with the methods for generating a vascular progenitor cell, or separately, e.g., starting with a hemangioblast-like cell or vascular progenitor cell. The purpose for differentiation is to predictively assess whether cells derived from progenitors possess positive or negative properties related to health or future health of the individual. For example, circulating hemangioblast cells may be purified and differentiated into hematopoietic stem cells in vitro. The differentiated cells are then tested for ability to generate neutrophils in response to G-CSF. If this ability is reduced then one predicts an abnormality in the subject and clinically tries to correct this through changes in lifestyle or through medical intervention. Methods for generating ECs include culturing a vascular progenitor cell in a solution appropriate for EC growth (e.g., EC growth media) and allowing the vascular progenitor cell to form an EC. In some aspects, the EC growth media includes endothelial cell growth factor, and the cells can optionally be grown in the presence of collagen. In some aspects, the vascular progenitor cell is cultured in EC growth media between 6 and 20 days, e.g., at least 6, 8, 10, 12 or 14 days (i.e., the course or duration of EC differentiation). In some aspects, the vascular progenitor cell is cultured in EC growth media for about 10-14 days. In some aspects, the ECs are cultured and maintained for multiple passages, e.g., for more than 1 or 2 months. As will be understood by one of skill in the art, the cells can be split and the cell media changed periodically during the course of culturing. One of skill will also appreciate that the method is typically carried out with a plurality of vascular progenitor cells, to form a population of cells that, over the course of differentiation, includes increasing numbers of ECs. In some aspects, the percentage of ECs in the population of cells increases to about 50-100% over the course of differentiation, e.g., more than 65, 75, 80, 85, 90, 95 or higher percentage ECs. In some aspects, the ECs can be further separated from the population using the methods described herein and prepared for storage (e.g., in freezing media) or therapeutic application. EC cell surface markers that can be used for separation include VE-cadherin, endoglin (CD105), vWF, and Z01 (tight junction protein), though negative selection can also be used (e.g., alpha-SMA or other non-endothelial cell marker). In some embodiments of the invention, smooth muscle cells (SMCs), and/or progenitors of smooth muscle cells are assessed. Methods for generating SMCs include culturing a vascular progenitor cell in a solution appropriate for SMC growth (e.g., SMC growth media) and allowing the vascular progenitor cell to form an SMC. In some aspects, the SMC growth media includes an SMC growth factor, and the cells can optionally be grown in the presence of collagen. In some aspects, the vascular progenitor cell is cultured in SMC growth media between 6 and 20 days, e.g., at least 6, 8, 10, 12 or 14 days (i.e., the course or duration of SMC differentiation). In some aspects, the vascular progenitor cell is cultured in SMC growth media for about 10-14 days. In some aspects, the SMCs are cultured and maintained for multiple passages, e.g., for more than 1 or 2 months. As will be understood by one of skill in the art, the cells can be split and the cell media changed periodically during the course of culturing. One of skill will also appreciate that the method is typically carried out with a plurality of vascular progenitor cells, to form a population of cells that, over the course of differentiation, includes increasing numbers of SMCs. In some aspects, the percentage of SMCs in the population of cells increases to about 50-100% over the course of differentiation, e.g., more than 65, 75, 80, 85, 90, 95 or higher percentage SMCs. In some aspects, the SMCs can be further separated from the population using the methods described herein and prepared for storage (e.g., in freezing media) or therapeutic application. SMC cell surface markers that can be used for separation include alpha-SMA and calponin, though again, negative selection using an non-SMC marker can be applied. Methods for generating a hematopoietic cell (e.g., erythrocyte (CFU-E), granulocyte-macrophage (CFU-GM), or macrophage (CFU-M)) include culturing a vascular progenitor cell in a solution or semisolid matrix appropriate for hematopoietic cell growth (e.g., hematopoietic cell growth media or matrix) and allowing the vascular progenitor cell to form a hematopoietic cell. In some aspects, the hematopoietic cell growth media includes at least one hematopoietic cell growth factor (e.g., stem cell factor (SCF), GM cell stimulating factor (GM-CSF), IL-3, EPO). In some aspects, the vascular progenitor cell is cultured in hematopoietic cell growth media between 10 and 30 days, e.g., at least 10, 12 14, 16, 18, 20, 22, or more days (i.e., the course or duration of hematopoietic cell differentiation). In some aspects, the vascular progenitor cell is cultured in hematopoietic cell growth media for about 14-21 days. In some aspects, the hematopoietic cells are cultured and maintained for multiple passages, e.g., for more than 1 or 2 months. As will be understood by one of skill in the art, the cells can be split and the cell media changed periodically during the course of culturing. One of skill will also appreciate that the method is typically carried out with a plurality of vascular progenitor cells, to form a population of cells that, over the course of differentiation, includes increasing numbers of hematopoietic cells. In some aspects, the hematopoietic cells can be further separated from the population by picking colonies, or using the methods described herein and prepared for storage (e.g., in freezing media) or therapeutic application. CD45 can be used for separation based on cell surface markers, but morphology of the hematopoietic cell colony is another useful separation tool. Additional information regarding cell culture techniques can be found, e.g., in Picot (2005) Human Cell Culture Protocols; Piper (1990) Cell Culture Techniques in Heart and Vessel Research; and Mather (2008) Stem Cell Culture, vol. 86, Meth. Cell Biol. Reagents and protocols for cell culture can also be found commercially, e.g., from Invitrogen, Gen-Probe, Lonza, and Clonagen. In some embodiments the correlation between cytokines in biological fluid and regenerative cells is made. Cytokines can be differentiated by those with an essentially pro-inflammatory mode of action, including tumor necrosis factor (TNF-alpha), interleukin-12, IL-18 and interferon gamma from those with anti-inflammatory mode of action, including IL-4, IL-10, IL-13 and the endogenous IL-1 receptor antagonist IL-1ra. In response to the local milieu of cytokines, CD4.sup.+ cells differentiate into the Th1 (pro-inflammatory) or Th2 (anti-inflammatory) lineage. Among the principal inducers of the Th1 and Th2 cells are IL-12 and IL-10, respectively. Cytokines involved in the Th1 process include IL-2, IFN-gamma and TNF, while those involved in the Th2 process include IL-3, IL-4, IL-5, IL-6, IL-10 and IL-13. Over 30 major members of the interleukin family have been identified, the majority of which play a role in atherogenesis. Specifically, they have been attributed to primarily anti-atherogenic (IL-1ra, IL-9, IL-10, IL-11) and pro-atherogenic (IL-1, IL-2, IL-6, IL-18) properties. Modulating these interleukins represent the most readily applicable approach to immunotherapy in atherosclerosis. It is believed that gut bacteria initiate an inflammatory response when epithelium TLRs recognize non-commensal microbial motifs and this cytokine signal may translate to increased risk of atherosclerosis. The corollary of this response is that commensal microflora are required to maintain gut homeostasis through the recognition of their non-inflammatory motifs by TLRs. Accordingly in some embodiments of the invention, elevated inflammatory cytokine levels and reduced anti-inflammatory cytokine levels are used to predict the eventual occurrence of a pathological health situation. In such a scenario, levels of stem cells and/or regenerative cells are further assessed, if these are also reduced, an increase in urgency of addressing and/or correcting the disbalance is needed. One way of correcting said disbalance is the utilization of various anti-inflammatory approaches. Such approaches would include, for example, the use of probiotics. In some embodiments probiotics are utilized alone, in others together with various enzymes. Various probiotics may be utilized, for example, probiotics of the genus Lactobacilli. Lactobacilli (i.e., bacteria of the genus Lactobacillus, hereinafter “L.”), including L. acidophilus, L. casei, L. fermentum, L. saliva roes, L. brevis, L. leichmannii, L. plantarum, L. cellobiosus, L. reuteri, L. rhamnosus, L. GG, L. bulgaricus, and L. thermophilus. According to other particular embodiments of this invention, the probiotic is chosen from the genus Bifidobacteria. Bifidobacteria also are known to exert a beneficial influence on human health by producing short chain fatty acids (e.g., acetic, propionic, and butyric acids), lactic, and formic acids as a result of carbohydrate metabolism. Non-limiting species of Bifidobacteria include B. angulatum, B. animalis, B. asteroides, B. bifidum, B. bourn, B. breve, B. catenulatum, B. choerinum, B. coryneforme, B. cuniculi, B. dentium, B. gallicum, B. gallinarum, B indicum, B. longum, B. magnum, B. merycicum, B. minimum, B. pseudocatenulatum, B. pseudolongum, B. psychraerophilum, B. pullorum, B. ruminantium, B. saeculare, B. scardovii, B. simiae, B. subtile, B. thermacidophilum, B. thermophilum, B. urinalis, and B. sp.

In one particular embodiment probiotics are administered as follows: 1 gram/ml=400 mg of bacteria or (4 billion CFU)+600 mg other components (echinacea 100 mg+500 ml paste emulsion), wherein, 100 mg/ml of bacteria=1 billion CFU and the proportions of the 100 mg/ml: are Actinomyces naeslundii-30%—early bio-film colonizer, Actinomyces odontolyticus—30%—early bio-film colonizer, Streptococcus thermophilius 20% common probiotics/biofilm colonizer, Lactobaccilus brevis-10% and Lactobacilius plantarum —10%. In this specific embodiment, for 2 mouth trays, 2-3 ml of gel per tray is administered. One of skill in the art will realize that specific volumes will depend on size of the arch, In one embodiment, phytoestrogens are added to the composition to enhance probiotic growth as well as to inhibit gingival inflammation. Phytoestrogens for embodiments of this invention include, but are not limited to, isoflavones, stilbenes, lignans, resorcyclic acid lactones, coumestans, coumestrol, equol, and combinations thereof. Sources of suitable phytoestrogens include, but are not limited to, whole grains, cereals, fibers, fruits, vegetables, black cohosh, agave root, black currant, black haw, chasteberries, cramp bark, dong quai root, devil's club root, false unicorn root, ginseng root, groundsel herb, licorice, liferoot herb, motherwort herb, peony root, raspberry leaves, rose family plants, sage leaves, sarsaparilla root, saw palmetto berried, wild yam root, yarrow blossoms, legumes, soybeans, soy products (e.g., miso, soy flour, soymilk, soy nuts, soy protein isolate, tempen, or tofu) chick peas, nuts, lentils, seeds, clover, red clover, dandelion leaves, dandelion roots, fenugreek seeds, green tea, hops, red wine, flaxseed, garlic, onions, linseed, borage, butterfly weed, caraway, chaste tree, vitex, dates, dill, fennel seed, gotu kola, milk thistle, pennyroyal, pomegranates, southernwood, soya flour, tansy, and root of the kudzu vine (pueraria root) and the like, and combinations thereof.

In some embodiments of the invention, probiotics are utilized in conjunction with EPC quantification to treat conditions listed below. Probiotics stimulates Treg [151-165], Treg inhibit GVHD [166-183]. Probiotics inhibit the chronic inflammation [184-186], chronic inflammation causes restenosis [187-189]. Disc degeneration is an inflammatory process [190]. Probiotics inhibit the chronic inflammation [184-186]. There is some evidence that DDD is associated with infection of Propionibacterium acnes inside of the disc [191-193]. There is evidence that probiotics can suppress growth of Propionibacterium acnes [194]. Spinal cord injury associated with dysbiosis [195-198], probiotics shown effective in animal model of spinal cord injury [199, 200]. Liver failure associated with dysbiosis [201, 202]. Liver failure associated with inflammation [203-205]. Probiotics inhibit progression of liver failure [206, 207]. Heart failure associated with dysbiosis [208-210]. Fixing dysbiosis reduces hypertension Autism associated with inflammatory profile [211-239], and reduced anti-inflammatory production [240, 241], autistic kids have dysbiosis [242-251], probiotics have positive effect in animal models [252-255].

It is well known to those having ordinary skill in the art that the edible gel mixes and edible gels may be prepared using other ingredients in addition to said prebiotic and/or probiotic mixtures or the composition comprising a compound of formula (1) and the gelling agent. Non-limiting examples of other ingredients for use in particular embodiments include a food acid, a salt of a food acid, a buffering system, a bulking agent, a sequestrant, a cross-linking agent, one or more flavors, one or more colors, and combinations thereof. Non-limiting examples of food acids for use in particular embodiments include citric acid, adipic acid, fumaric acid, lactic acid, malic acid, and combinations thereof. Non-limiting examples of salts of food acids for use in particular embodiments include sodium salts of food acids, potassium salts of food acids, and combinations thereof. Non-limiting examples of bulking agents for use in particular embodiments include raftilose, isomalt, sorbitol, polydextrose, maltodextrin, and combinations thereof. Non-limiting examples of sequestrants for use in particular embodiments include calcium disodium ethylene tetra-acetate, glucono delta-lactone, sodium gluconate, potassium gluconate, ethylenediaminetetraacetic acid (EDTA), and combinations thereof. Non-limiting examples of cross-linking agents for use in particular embodiments include calcium ions, magnesium ions, sodium ions, and combinations thereof. In one embodiment a sweetener is added to said composition, said sweeter is selected from the group consisting of sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, dulcoside A, dulcoside B, rubusoside, stevia, stevioside, mogroside IV, mogroside V, Luo han guo, siamenoside, monatin and its salts, curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, steviolbioside and cyclocarioside I, sugar alcohols, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs) and combinations thereof. Furthermore, prebiotics may be added to the composition useful for modulation of oral microbiome, said prebiotics include D-turanose, D-melezitose, D-lactitol, myo-inositol, N-acetyl-D-mannosamine and mixtures thereof, wherein the amount of saccharide prebiotic in the composition promotes the growth of beneficial endogenous bacteria, e.g., wherein the beneficial endogenous bacteria are one or more species selected from the group consisting of Streptococcus mitis, Streptococcus salivarius, Streptococcus sanguinis, Actinomyces viscosus, Veillonella parvula, Streptococcus gordonii, Capnocytophaga sputigena and Actinomyces naeslundii. In one embodiment gelling agents are added to said composition useful for modulating oral microbiome, said gelling agents include gelling and/or thickening agent which selected from the group consisting of polyvinylpyrrolidone, carboxymethylcellulose, carboxypropylcellulose, carboxypolymethylene, hydroxymethylcellulose, sodiumcarboxymethylcellulose and other cellulose salts, carrageenan, gum karaya, xanthan gum, guar gum, gum Arabic, gum tragacanth, carboxyvinyl polymers, poloxomer, silica compounds, starches, hydroxyethylpropylcellulose, hydroxybutylmethylcellulose, hydroxypropylmethylcellulose and mixtures thereof. In one embodiment, the gelling agent is carboxymethylcellulose and is present in an amount of from about 0.001% by weight to about 50% by weight of the composition. In one embodiment agents inhibiting proteolytic activity are added to said composition, such as inhibitors of matrix metalloproteases. In another embodiment, a vehicle in the preparation of the compositions is added to prevent drying of teeth, especially when administration is performed overnight or for extended periods of time. For example, the humectants glycerin and polyethylene glycol admixed with water may be employed in preparing a vehicle for the peroxide-based component of the whitening compositions, to which other ingredients described above may be added. Preferably, however, care is taken to exclude water or moisture from the whitening compositions during and after preparation to avoid premature activation of the peroxide component. In another typical example preparation the peroxide whitening composition may be prepared a paste at ambient temperature by the use of anhydrous glycerin with a peroxide compound, such as the desired amount of carbamide peroxide, along with adjuvants and excipients, such as chelating agents, sweeteners and flavoring premixed with water until all solids are dissolved. Thickeners and/or stabilizers are separately processed and mixed until a uniform mixture is obtained, and then both admixtures are combined and stirred until a desired gel forms. Abrasives, such as calcium phosphate or various types of silica including hydrated silica compounds having a variety of particle sizes, ranging from submicron sizes to several microns, may then be added and the mixture stirred and/or processed until a uniform dispersion is obtained. While these methods are merely exemplary of some preferred embodiments of whitening composition preparation, the skilled artisan will undoubtedly find many other suitable methods without undue effort, depending upon the particular nature of the constituents employed. In some embodiments, enhancement of anti-inflammatory cytokines and/or circulating endothelial progenitor cells and/or regenerative cells is performed by administration of probiotics. In some embodiments administration of a blend of probiotics is performed; specifically, Bifidobacterium infantis, Bifidobacterium bifidum, Lactobacillus acidophilus, Lactobacillus salivarius, Lactobacillus plantarum, Lactobacillus rhamnosus, Bifidobacterium longum, Lactobacillus casei, Lactobacillus paracasei, in combination with a blend of digestive enzymes; specifically, amylase, glucoamylase, lipase, bromelain, maltase, lactase, hemicellulase, xylanase, papain, and invertase. Identifying a patient in need can be done by any conventional detection method, non-exclusively including blood tests, or identifying and assessing risk factors for cardiovascular disease, such as smoking, drinking, lack of exercise, weight of patient, age, family history, etc. probiotic digestive enzyme dietary supplement and a method of use for humans or other animals. The “probiotic composition” (which may also exist as a drug or pharmaceutical) may comprise at least two ingredients. The two ingredients may include at least one probiotic ingredient. For example, the composition may include at least two different probiotic ingredients or at least one probiotic ingredient and at least one digestive enzyme ingredient, or other ingredients in various combinations. In addition, the composition may be substantially, if not completely, devoid of artificial flavors, colorings or preservatives. Further, the supplement may be developed for human or other animal consumption by swallowing or other ingestion technique. In a situation where the composition is developed for consumption by swallowing, the composition may be enclosed within a capsule or other form known to facilitate swallowing. The terms probiotics, digestive enzymes and dietary supplements have generally accepted definitions. For example, probiotics may be defined as live microorganisms thought to be healthy for the host organism; digestive enzymes may be defined as enzymes that break down polymeric macromolecules into their smaller building blocks in order to facilitate their absorption by the body; dietary supplements may be defined as a preparation intended to supplement the diet and provide nutrients that may be missing or may not be consumed in sufficient quantities in a human's diet. The probiotic ingredients of the composition may be present in an effective dose. For example, at the time of manufacture, the probiotic ingredients may total at least 6×1 Q9 colony forming units (cfu) and may include at least 13×1Q9 cfu of probiotics or more. In a preferred aspect, the probiotic ingredients total at least 13×1Q9 cfu of probiotics. In a more preferred aspect the probiotic ingredients total at least 14×1Q9 cfu of probiotics. A colony forming unit (cfu) is generally accepted as a measure of viable bacterial or fungal numbers. Such quantity of probiotic ingredient may facilitate providing a consumer with an effective dose of probiotics at the time of ingestion, as the inventor has realized that probiotics may be destroyed during storage due to undesirable environments (e.g., temperature extremes) and other reasons. The probiotic ingredients may comprise a probiotic blend including one or more of the following: Lactobacillus rhamnosus GG, Lactobacillus acidophilus, Lactobacillus casei, Lactobacillus paracasei, Lactobacillus plantarum, Lactobacillus salivarius, Bifidobacterium infantis, Bifidobacterium longum, Bifidobacterium bifidum. In a preferred aspect the composition includes at least one probiotic from each of the strains listed above. Each probiotic ingredient present in the composition may be present in any desired quantity. In one aspect each probiotic ingredient of the composition may be present in an amount at least between 5×1os cfu to 1.5×1Q9 cfu. In a further aspect each probiotic ingredient of the composition may be present in an amount equal to or greater than 1×1Q9 cfu, and in a preferred aspect when combined the nine probiotic ingredients may total as much as, or more than, 13×1Q9 cfu of probiotic ingredients. Preferably the amounts are equal to or are more than 14×1Q9 cfu. In the example, these quantities may be measured at the time of manufacture.

The inventor appreciates that simply introducing a probiotic or probiotics into the GI tract as done in prior instances is not effective due to otherwise poor or undesirable placement of the ingredient within the system and/or lack of an effective dose due to the degradation of the living probiotic ingredient and/or lack of the variety and nature of a desired or sufficient strain or strains of probiotic and/or lack of use of the probiotic blend in combination with the digestive enzyme supplement or supplemental blend (see below regarding enzymes). Further, as the inventor appreciates that lactobacillis acidophilis is a prominent strain of probiotic in the small intestine, and Bifidobacterium bifidum is a prominent strain of probiotic in the large intestine, for instance, it is advantageous to have those supplemental ingredients (and other of the respective supplemental probiotic ingredients noted above and the digestive enzymes noted below) introduced into the GI tract at the appropriate or preferred locations (and in effective amounts). Use of a capsule, such as a vegetable or other capsule that does not immediately release the contents therein (for instance, the capsule delays release beyond the stomach), has a benefit for the positioning of the ingredients within or throughout the GI tract. Use of a blister pack (or other sealing mechanism) for storing the capsule assists in preserving the potency of the ingredients such that the combination of the composition with the capsule in a protected blister package assists with appropriate and effective delivery (location and potency). Further, the particular blend and strains of the respective ingredients, and in the various amounts, have been established by the inventor for desired impact and appropriate delivery. Digestive enzymes of the composition may be present in an effective dose to supplement existing quantities of enzymes and improve digestion of ingested food and absorption of the nutrients within the ingested food. The digestive enzyme ingredients may comprise any enzyme that is useful in the digestion of ingested food. For example, inventors has developed a particularly effective blend of digestive enzymes comprising some or all of the following: Amylase, Protease, Lipase, Hemicullelase, Invertase, Lactase, Papain, Glucoamylase, Xylanase, and Maltase.

A capsule may enclose the composition to facilitate increasing the shelf-life of the composition, swallowing of the composition, timing a release of the composition after ingestion and other considerations. The capsule may be a gelatin capsule, vegetable-based (e.g., vegetable cellulose) capsule or other type of capsule. If the capsule is a vegetable-based capsule, the capsule may facilitate releasing the probiotics at a desirable location within the digestive tract. Preferably the capsule is a vegetable-based capsule. An exemplary embodiment of the composition includes a probiotic blend and a digestive enzyme blend enclosed within a vegetable cellulose capsule, as seen in FIG. 2. The probiotic blend includes 13×1Q9 cfu (130 mg) of probiotics consisting of the following species: Lactobacillus rhamnosus GG, Lactobacillus acidophilus, Lactobacillus casei, Lactobacillus paracasei, Lactobacillus plantarum, Lactobacillus salivarius, Bifidobacterium infantis, Bifidobacterium longum, Bifidobacterium bifidum. Preferably each probiotic species is present in quantities of at least 1×1 Q9 cfu. In one aspect the digestive enzyme blend includes 750 mg of digestive enzymes consisting of the following: amylase, protease, lipase, hemicellulase, invertase, lactase, papain, glucoamylase, xylanase and maltase.

Protecting the composition after manufacture is particularly important as at least the probiotic ingredients may be sensitive to variations in environmental conditions. To facilitate protection of the composition, capsules comprising the composition may be and are preferably stored in blister packs. That is, the blister packs may seal the capsule from a surrounding environment and thus, extend the life of the effective ingredients of the composition.

The method of using the composition may be used as desired by consumers of the composition. A particularly advantageous program may be to take a single capsule of the composition on a daily basis. Continuous daily use of the composition may result in greater comfort throughout the digestive tract, which may further result in increased energy and general good health of a consumer's body.

Lactobacillus is a genus within the lactic acid bacteria group, named because the majority of its members convert lactose and other sugars to lactic acid. Lactobacillus may naturally be present in the gastrointestinal tract and other areas of the human body. Lactobacillus is used for treating and preventing diarrhea, including infectious types such as rotaviral diarrhea in children and traveler's diarrhea. It is also used to prevent and treat diarrhea associated with using antibiotics. Other benefits of lactobacillus may include relief from general digestion problems; irritable bowel syndrome (IBS); Crohn's disease; inflammation of the colon; and infection with Helicobacter pylori, the type of bacteria that causes ulcers. Numerous strains of Lactobacillus may be utilized within the composition as exemplary set forth below. Bactobacillus rhamnosus GG. When administered orally, L. rhamnosus GG adheres to the mucous membrane of the intestine and may help to restore the balance of the GI micro flora; promote gut-barrier functions; diminish the production of carcinogenic compounds by other intestinal bacteria; and, activate the innate immune response and enhance adaptive immunity, especially during infections. Lactobacillus acidophilus. Within the digestive system, L. acidophilus has been observed as preventing the growth of fungus; thus, helping to prevent infections. Further, L. acidophilus may facilitate lactose digestion in lactose-intolerant subjects and may facilitate the re-colonization of probiotics in the gastrointestinal tract to achieve normal intestinal flora levels.

Lactobacillus casei. L. casei is considered beneficial for the digestive process. It has a wide temperature and pH range meaning it can withstand the acidic environment of the gut. It also promotes L. acidophilus which produces the enzyme amylase. This enzyme assists a person's body in the digestion of carbohydrates, which can help reduce, relieve or prevent conditions such as constipation, lactose intolerance and possibly irritable bowel syndrome. Lactobacillus paracasei. L. paracasei is a strain of flora (i.e., bacteria) that helps to calm digestive upsets and assists other strains of bacterium. As well, L. paracasei may improve the absorption of nutrients and lipids in the gut. Lactobacillus salivarius. L. salivarius has been shown to improve bleeding gums, tooth decay, bad breath, thrush and canker sores. In addition, L. salivarius breaks down proteins and produces B vitamins, enzymes and lactic acid. Lactobacillus plantarum. L. plantarum may create a healthy barrier in a person's colon to keep dangerous bacteria from penetrating the lining of a person's intestines and entering a person's blood stream. [Para 44] Bifidobacterium and Species Thereof. Bifidobacterium generally reside in the colon and are one of the major genera of bacteria that make up the gut flora. This probiotic may be used to relieve and treat intestinal disorders; may assist in digestion; may be associated with lowering occasions of allergies; and may assist in preventing certain tumor growths. Moreover, the benefits of Bifidobacterium include regulation of intestinal microbial homeostasis, inhibition of pathogens and harmful bacteria that colonize or infect, or both, the gut mucosa, modulate local and systemic immune responses, assist in producing vitamins, and help bioconvert a number of dietary compounds into bioactive molecules. Numerous strains of Bifidobacterium may be utilized within the composition as exemplarily set forth below. Bifidobacterium longum. B. longum has a high affinity for intestinal colonization. Generally, this probiotic assists in improving the intestinal environment, which may lead to better regularity of bowel movements. Moreover, B. longum assists in maintaining a normal digestive tract, inhibits the growth of harmful bacteria and boosts the immune system. Bifidobacterium bifidum. B. bifidum helps keep the digestive system running smoothly, blocks the growth of harmful bacteria, and boosts the immune system. Bifidobacterium Infantis 3 5624 (or other strains). B. infantis may help relieve many of the symptoms associated with irritable bowel syndrome (IBS) in women, including diarrhea and constipation. Many enzymes and molecules are utilized to break down all the food we eat. In fact, the process of digestion, or the breaking down of large pieces of food, is complex and involves several enzymes, each with a specific function. There is a range of enzymes in our digestive system and each enzyme functions to keep us healthy through breaking down “big” pieces of ingested food into small absorbable molecules. The food entering our stomach is made easier to digest by the act of chewing and by the addition of moisture from our saliva and the liquid we drink. The stomach contains several enzymes that work together to partially break down ingested food substances, examples follow: Amylase. Amylase is an enzyme found in our saliva and in the enzyme blend released from the pancreas. It functions primarily as astarch-dissolving enzyme that takes starch in our food and breaks it down into simple sugars which can be more easily absorbed. Without an enzyme such as Amylase to break down starch, the bacteria residing in our colon would use the starch for food, resulting in bacterial overgrowth, bloating and gas. Preferred amounts of amylase for the teachings herein include 1.33 mg or between 0.5-2.5 mg. Lipase. This enzyme works throughout the digestive process to break down the fats in our diet. It works with the bile salts excreted from the liver to emulsify and digest long fat molecules. Without lipase, fat would pass quickly through our system, resulting in the possibility of diarrhea and sometimes leakage from the lower bowel. In addition, the human body relies on certain essential fatty acids that can only be derived from food and Lipase is used to cultivate those fatty acids. Without Lipase, the human body cell structures cannot function normally and humans would also suffer from extremely dry skin and hair. Preferred amounts of lipase for the teachings herein include 25 mg or between 18-32 mg.Protease. Protease is the general term for an enzyme that breaks down proteins. Proteins are molecules that make up much of our living tissue, including our muscles and our internal systemic enzymes. Certain proteins can only be provided through our food and Protease assists in breaking down our food to cultivate those proteins. If the human body has an inadequate means of cultivating proteins; humans would suffer from what is known as “protein malnutrition”. Proteins are broken down in several steps. As most proteins are not simply long, skinny molecules, but rather coil-like, they must be “unraveled”. Much of this unraveling is done in the acidic environment of the stomach. Generally, proteins do not tolerate an acidic environment unless specifically designed to do so. Once unraveled, proteases break down the pieces of the protein into amino acids that are easily absorbed into the human body. Much of the body wouldn't be able to function properly without essential amino acids from absorbable protein. A preferred protease that can be used with each embodiment herein relating to a protease is bromelain. teachings herein is bromelain, which refers to either of two protease enzymes extracted from the plants of the family Bromeliaceae. Preferred amounts of protease, such as bromelain, that can be used in the teachings herein include 18 mg or between 13-23 mg. Maltase. Maltase is an enzyme that breaks down a specific sugar that is ingested by humans. The particular sugar is malt sugar, which is often found in malt liquor and other malted foods. Preferred amounts of maltase, that can be used in the teachings herein include 10 mg or between 8-12 mg. lnvertase. lnvertase is also an enzyme that breaks down a specific sugar ingested by humans. That particular sugar is sucrose or table sugar. Those of us with a high sugar intake are in particular need of this enzyme. If invertase cannot do its job, the bacteria in our gut are left to attack the ingested sugars. Stomach cramps, bloating and gas can result if sugar-digesting enzymes are inadequate. Preferred amounts of invertase, that can be used in the teachings herein include 18 mg or between 13-23 mg. Lactase. Lactase is another specific enzyme that breaks down sugars, particularly the sugar found in dairy products. Without lactase, our ability to drink milk or consume other dairy products would be greatly impaired. As with many enzymes, you don't have to be born with intolerance to lactose to have insufficient lactase levels. Any individual who quits consuming dairy products for a period of time may find that when they begin drinking milk again, they are suddenly intolerant. This is because the body gradually “forgets” to make an enzyme that isn't getting used much. In this case, lactase supplementation may become necessary later in life even though an individual has not previously had a problem with lactose intolerance. Preferred amounts of lactase that can be used in the teachings herein include 9.5 or between 8.0-11 mg. Papain. Papain, also known as papaya proteinase I, works in the digestive system to break up specific segments of proteins into smaller amino acids. Specifically, papain is beneficial for breaking down tough meat fibers. Preferred amounts of papain that can be used in the teachings herein include 1.7 mg or between 0.5-3 mg. Hemicellulase. Hemicellulase is an enzyme that is vital to the digestion of plant material. Plant cell walls are made from cellulose and are often difficult to digest. Poor plant digestion leaves an excess of roughage that is eaten by bacteria in the colon. The end result is gas and sometimes intolerance to raw vegetables. Hemicellulase keeps this intolerance from happening and maximizes the nutrients that can be absorbed from raw vegetables. Preferred amounts of Hemicellulase that can be used in the teachings herein include 8 mg—or between 5-11 mg. Glucoamylase. Glucoamylase is a saccharide digestive enzyme. It is found mostly in mucosa and its function is to assure the breakdown of maltose into glucose molecules. Preferred amounts of Glucoamylase that can be used in the teachings herein include 50 mg or between 30-70 mg. Xylanase is a group of enzymes that break down components of the cell wall matrix of plants (fiber), such as hemicellulose. Although xylanase is not produced by humans, it is present in fungi from which it may be used for the degradation of plant matter into usable nutrients.

In one further aspect of the invention the composition may include at least one ingredient of each of the lactobacillus varieties noted above for at least 3×1Q9 colony forming units (at time of manufacture assuming half billion cfu per probiotic; and preferably 6×1Q9 colony forming units assuming 1 billion cfu per probiotic at time of manufacture), together with a digestive enzyme and contained in a vegetable-based capsule. Preferably the capsule is stored in a blister pack. Preferred amounts of Xylanase that can be used in the teachings herein include 3.9 mg or between 2-6 mg. In one further aspect of the invention the composition may include at least one ingredient of each of the lactobacillus varieties and at least one ingredient of each of the bifidobacterium varieties noted above for at least 4.5×1Q9 colony forming units (at time of manufacture assuming half billion cfu per probiotic; and preferably 9×1Q9 colony forming units assuming 1 billion cfu per probiotic at time of manufacture), together with a digestive enzyme and contained in a vegetable-based capsule. Preferably the capsule is stored in a blister pack. In yet a further aspect of the invention the composition may include at least one of the probiotics of the lactobacillus variety and at least one probiotic of the bifidobacterium variety together with a digestive enzyme and contained in a vegetable-based capsule for at least 2×109 colony forming units. Preferably the capsule is stored in a blister pack. More preferably the blend includes at least some additional probiotic ingredients as noted above and at least 6×1Q9 colony forming units assuming 1 billion cfu per probiotic at time of manufacture. In a further and preferred aspect, the composition may include, for instance, Lactobacillus acidophilus, Lactobacillus rhamnosus CG, Bifidobacterium lnfantis, and Bifidobacterium bifidum, together with a digestive enzyme and contained in a vegetable-based capsule. In a further preferred aspect the foregoing composition may include others from the above list of probiotics for at least 9×1Q9 colony forming units, and stored in a blister pack or other sealed package. In a further preferred aspect the foregoing composition may include others from the above list of probiotics for at least 13×1Q9 colony forming units, and stored in a blister pack or other sealed package. In a further aspect the composition may include a greater amount of lactobacillus probiotic as compared to bifidobacterium probiotic. In some embodiments, the composition(s) of the invention, including probiotics, and probiotics together with enzymes are administered in the form of a nutraceutical. Nutraceuticals, whether in the form of a liquid extract or dry composition, are edible and may be eaten directly by humans or mammals. Said nutraceuticals are preferably provided to humans in the form of additives or nutritional supplements for example they may be administered in the form of tablets of the kind sold in health food stores, or as ingredients in edible solids, more preferably processed food products such as cereals, breads, tofu, cookies, ice cream, cakes, potato chips, pretzels, cheese, and in drinkable liquids such as beverages such as milk, soda, sports drinks, and fruit juices. Thus, in one embodiment a method is provided for enhancing the nutritional value of a food or beverage by intermixing the food or beverage with a nutraceutical in an amount that is effective to enhance the nutritional and probiotic or immune modulatory and/or cancer therapy augmentative value of the food or beverage. In one embodiment, a flavoring agent is added. Preferred flavoring agents include sweeteners such as sugar, corn syrup, fructose, dextrose, maltodextrose, cyclamates, saccharin, phenyl-alanine, xylitol, sorbitol, maltitol, and herbal sweeteners such as Stevia. Examples of foods into which probiotics useful for the practice of the invention can be incorporated into include soft drinks, a fruit juice or a beverage comprising whey protein, health teas, cocoa drinks, milk drinks and lactic acid bacteria drinks. Probiotic bacteria may be administered together with agents known to enhance efficacy and retention of probiotics, including In a further embodiment of the present invention various extracts and plant powders are incorporated into our compositions, depending on the desired properties according to the end use of said compositions. These compositions according to the present invention can be characterized in that in addition to the discussed prebiotics and phytosterols and lecithins the said further plant extracts or powders are one or more of those of Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Whitania somnifera (ashwagandha, Indian ginseng), Panax japonicus (Japanese ginseng), Panax vietnamensis (Vietnamese ginseng), Panax trifolius, Panax pseudoginseng, Panax notoginseng, Malpighia glabra (acerola), Ylex paraguayiensis (Yerba mate), Astragalus membranaceus (astragalus), Stevia rebaudiana (stevia), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola (and other Cistanche sp.), Codonopsis pilosula (“poor man's ginseng.”), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), Cordyceps sinensis (Cordycaps), Lentinula edodes (shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Trametes versicolo, Coriolus versicolor (Turkey tails), Schizophyllum commune (Split gill), Inonotus obliquus (Cinder conl), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum). These plant extracts and plant powders are capable to potentiate the bioactivity of these compositions based on prebiotics, phytosterols, lecithins, vitamins and minerals. In given cases it also adds other prebiotics to the aforementioned prebiotic mixtures. These can result in more pronounced bioactivities as prebiotics and also in the chosen other bioactivity directions.

The various probiotic, and probiotic/enzyme mixtures described herein are intended for human consumption and thus the processes for obtaining them are preferably conducted in accordance with Good Manufacturing Practices (GMP) and any applicable government regulations governing such processes. Especially preferred processes utilize only naturally derived solvents. In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label must clearly state that the product is intended to be used to manage a specific medical disorder or condition. An example of a medical food is nutritionally diverse medical food designed to provide targeted nutritional support for patients with chronic inflammatory conditions. Active compounds of this product are for instance one or more of the compounds described herein. The present invention thus relates to the use of an immuno-modulating properties of probiotics as related to prevention and/treatment of pregnancy complications. Thus said probiotics can be used in the preparation of a medicament, a vaginal suppository, medical food or nutraceutical to induce immune tolerance or immune modulation.

In some embodiments, the compositions according to the present invention comprise prebiotic components selected from fructose polymers GF.sub.n and F.sub.m, either containing a glucose (G) end-group, or without this glucose end-group and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives. The fructose polymers of GF.sub.n or F.sub.m structures (G=glucose; F=fructose; n>2; m>2) are linear fructose polymers having either a glucose (G) and -group, or being without this glucose and -group. Oligofructoses are consisted of 3 to 10 carbohydrate units. Above that, chicory inulin contains 10 to 60 carbohydrate units, typically with 27 carbohydrates (fructoses with our without one glucose end-group and a fructose chain). Other plants may produce different fructans. These fructans are capable to increase the number of colonized and planktonic bacteria in the large intestine. This results in a change that those bacteria that are less advantageous or may turn dangerous are suppressed by the higher probiotic colony of bacteria. Depending on the chain length of these fructans or other prebiotics, they can be fermented by probiotic bacteria at different positions in the colon.

In some embodiments of the invention teaches detection of degeneration caused by chemotherapy. Through assessing the regenerative capacity of cellular components found in circulation, the invention provides a means of identifying what level of cancer therapy, such as chemotherapy may be administered without significantly affecting regenerative potential of the body. In other embodiment the approach described is utilized for determining the amount of radiation given during radiotherapy.

Broadly speaking, chemotherapy may be divided into the following classes: 1) Alkylating agents. These drugs kill cells that are not in cell cycle [256, 257], and include mustard gas derivatives such as Mechlorethamine, Cyclophosphamide, Chlorambucil, Melphalan [258], and Ifosfamide. Ethylenimines such as Thiotepa and Hexamethylmelamine, Alkylsulfonates such as Busulfan, Hydrazines. Triazines such as Altretamine, Procarbazine, Dacarbazine and Temozolomide. Nitrosureas such as Carmustine, Lomustine and Streptozocin. Metal salts such as Carboplatin, Cisplatin, and Oxaliplatin [259]. Alkylating agents are one of the original classes of chemotherapies that where historically developed [260, 261]. 2) Plant Alkaloids. These are chemotherapeutic drugs that are extracted from certain types of plants. The plant alkaloids are cell-cycle specific. This means they attack the cells during various phases of division. The vinca alkaloids are made from the periwinkle plant (catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also known as topoisomerase inhibitors, which are used in certain types of chemotherapy. Vinca alkaloids include Vincristine, Vinblastine and Vinorelbine. Taxanes include Paclitaxel and Docetaxel. Podophyllotoxins include Etoposide and Tenisopide. Camptothecan analogs include Irinotecan and Topotecan. 3) Antitumor Antibiotics. This type of chemotherapy is generated from species of the soil fungus Streptomyces. These drugs act during multiple phases of the cell cycle and are considered cell-cycle specific. Antitumor antibiotics include Anthracyclines such as Doxorubicin, Daunorubicin, Epirubicin, Mitoxantrone, and Idarubicin. Anthracyclines are generally a class of compounds that have the structural core of anthracene. They often are highly effective chemotherapeutics and therefore are used for the treatment of many cancers, including leukemias, lymphomas, breast, uterine, ovarian, bladder cancer, and lung cancers and are often used in childhood cancer treatment regimens. Some anthracycline drugs include doxorubicin, daunorubicin, idarubicin, and epirubicin. Although the exact mechanisms may yet to be validated, anthracyclines have been reported to work by inhibiting DNA and RNA synthesis; promoting free radical formation through redox cycling, with iron promoting the conversion of superoxide into hydroxyl radicals; inhibiting topoisomerases (e.g., topoisomerases II.alpha. and/or II.beta.); and evicting histones from open chromosomal areas. Chromomycins such as Dactinomycin and Plicamycin. Additional antitumor antibiotics include Mitomycin and Bleomycin. 4) Antimetabolites. This type of chemotherapy resembles normal substances within the cell. When the cells incorporate these substances into the cellular metabolism, they are unable to divide. Antimetabolites are cell-cycle specific. They attack cells at very specific phases in the cycle. Antimetabolites are classified according to the substances with which they interfere. Antimetabolites include the folic acid antagonist Methotrexate, the pyrimidine antagonists 5-Fluorouracil, Foxuridine, Cytarabine, Capecitabine, and Gemcitabine, the purine antagonists 6-Mercaptopurine and 6-Thioguanine, the adenosine deaminase inhibitors Cladribine, Fludarabine, Nelarabine and Pentostatin. 5) Topoisomerase Inhibitors. These chemotherapeutic agents that interfere with the action of topoisomerase enzymes (topoisomerase I and II). During the process of chemo treatments, topoisomerase enzymes control the manipulation of the structure of DNA necessary for replication. Ironotecan and topotecan are considered topoisomerase I inhibitors, whereas amsacrine, etoposide, etoposide phosphate, and teniposide are considered topoisomerase II inhibitors. 6) Alternative types of chemotherapy. Hydroxyurea is considered a ribonucleotide reductase inhibitor. Mitotane is considered an adrenocortical steroid inhibitor. Asparaginase and Pegaspargase are enzymatic types of chemotherapy. Estramustine is considered an antimicrotubule drug and members of the retinoid family of chemotherapies include Bexarotene, Isotretinoin, Tretinoin (ATRA). In one embodiment the number of circulating EPC are assessed prior to therapy, and during therapy. Various levels of EPC in circulation are utilized to guide the administration of therapy and to predict “danger zones” in which too much chemotherapy can lead to substantial reduction of regenerative activity without additional cancer suppressing activity. Specific non-limiting examples of chemotherapeutic agents are provided throughout the specification and include, for example, FOLFOX (a chemotherapy regimen for treatment of colorectal cancer, which comprises administration of folinic acid (leucovorin), fluorouracil (5-FU), and oxaliplatin) and FOLFIRI (a chemotherapy regimen for treatment of colorectal cancer, which comprises administration of folinic acid (leucovorin), fluorouracil (5-FU), and irinotecan), as well as administration of targeted monoclonal antibody therapy (e.g., bevacizumab, cetuximab, or panitumumab) alone or in combination with chemotherapeutic agents.

The term “chemotherapy cycle” is used herein to refer to a period of time between the initial administration of an anti-cancer agent and its repeat administration. For example, the cycle of the FOLFOX4 chemotherapy includes 14 days, wherein anti-cancer agents are administered only for the first 2 days of the cycle as follows: Day 1: oxaliplatin 85 mg/m.sup.2 IV infusion and leucovorin 200 mg/m.sup.2 IV infusion both given over 120 minutes at the same time in separate bags, followed by 5-FU 400 mg/m.sup.2 IV bolus given over 2-4 minutes, followed by 5-FU 600 mg/m.sup.2 IV infusion as a 22-hour continuous infusion; Day 2: leucovorin 200 mg/m.sup.2 IV infusion, followed by 5-FU 400 mg/m.sup.2 IV bolus given over 2-4 minutes, followed by 5-FU 600 mg/m.sup.2 IV infusion as a 22-hour continuous infusion. Similarly, the cycle of the FOLFIRI chemotherapy includes 14 days, wherein anti-cancer agents are administered only for the first 2 days of the cycle as follows: irinotecan (180 mg/m.sup.2 IV over 90 minutes) concurrently with folinic acid (400 mg/m.sup.2 [or 2.times.250 mg/m.sup.2] IV over 120 minutes), followed by fluorouracil (400-500 mg/m.sup.2 IV bolus) then fluorouracil (2400-3000 mg/m.sup.2 intravenous infusion over 46 hours). Bevacizumab is usually given intravenously every 14 days, although the frequency can be dose dependent (for example 5 mg/kg by intravenous infusion every two weeks or 7.5 mg/kg by intravenous infusion every three weeks). In colon cancer, it is given in combination with the chemotherapy drug 5-FU (5-fluorouracil), leucovorin, and oxaliplatin or irinotecan. One recommended dose and schedule for cetuximab is 400 mg/m.sup.2 administered intravenously as a 120-minute infusion as an initial dose, followed by 250 mg/m.sup.2 infused over 30 minutes weekly, preferably in combination with FOLFIRI.

Many chemotherapeutic agents are known in the art and include but are not limited to: methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, STI-571 or Gleevec.™. (imatinib mesylate), herbimycin A, genistein, erbstatin, and lavendustin A. taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostinsherbimycin A, genistein, erbstatin, and lavendustin ABCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, the anti-cancer agent can be, but is not limited to, a drug listed: Alkylating agents Nitrogen mustards: Cyclophosphamide Ifosfamide Trofosfamide Chlorambucil Nitrosoureas: Carmustine (BCNU) Lomustine (CCNU) Alkylsulphonates: Busulfan Treosulfan Triazenes: Dacarbazine Platinum containing Cisplatin compounds: Carboplatin Aroplatin Oxaliplatin Plant Alkaloids Vinca alkaloids: Vincristine Vinblastine Vindesine Vinorelbine Taxoids: Paclitaxel Docetaxel DNA Topoisomerase Inhibitors Epipodophyllins: Etoposide Teniposide Topotecan 9-aminocamptothecin Camptothecin Crisnatol mitomycins: Mitomycin C Anti-metabolites Anti-folates: DHFR inhibitors: Methotrexate Trimetrexate IMP dehydrogenase Mycophenolic acid Inhibitors: Tiazofurin Ribavirin EICAR Ribonuclotide reductase Hydroxyurea Inhibitors: Deferoxamine Pyrimidine analogs: Uracil analogs: 5-Fluorouracil Floxuridine Doxifluridine Ratitrexed Cytosine analogs: Cytarabine (ara C) Cytosine arabinoside Fludarabine Purine analogs: Mercaptopurine Thioguanine DNA Antimetabolites: 3-HP 2′-deoxy-5-fluorouridine 5-HP alpha-TGDR aphidicolin glycinate ara-C 5-aza-2′-deoxycytidine beta-TGDR cyclocytidine guanazole inosine glycodialdehyde macebecin II pyrazoloimidazole Hormonal therapies: Receptor antagonists: Anti-estrogen: Tamoxifen Raloxifene Megestrol LHRH agonists: Goserelin Leuprolide acetate Anti-androgens: Flutamide Bicalutamide Retinoids/Deltoids Cis-retinoic acid Vitamin A derivative: All-trans retinoic acid (ATRA-IV) Vitamin D3 analogs: EB 1089 CB 1093 KH 1060 Photodynamic therapies: Vertoporfin (BPD-MA) Phthalocyanine Photosensitizer Pc4 Demethoxy-hypocrellin A (2BA-2-DMHA) Cytokines: Interferon-.alpha. Interferon-.gamma. Tumor necrosis factor Angiogenesis Inhibitors: Angiostatin (plasminogen fragment) antiangiogenic antithrombin III Angiozyme ABT-627 Bay 12-9566 Benefin Bevacizumab BMS-275291 cartilage-derived inhibitor (CDI) CAI CD59 complement fragment CEP-7055 Col 3 Combretastatin A-4 Endostatin (collagen XVIII fragment) Fibronectin fragment Gro-beta Halofuginone Heparinases Heparin hexasaccharide fragment HMV833 Human chorionic gonadotropin (hCG) IM-862 Interferon alpha/beta/gamma Interferon inducible protein (IP-10) Interleukin-12 Kringle 5 (plasminogen fragment) Marimastat Metalloproteinase inhibitors (TIMPs) 2-Methoxyestradiol MMI 270 (CGS 27023A) MoAb IMC-1C11 Neovastat NM-3 Panzem PI-88 Placental ribonuclease inhibitor Plasminogen activator inhibitor Platelet factor-4 (PF4) Prinomastat Prolactin 16 kD fragment Proliferin-related protein (PRP) PTK 787/ZK 222594 Retinoids Solimastat Squalamine SS 3304 SU 5416 SU6668 SU11248 Tetrahydrocortisol-S tetrathiomolybdate thalidomide Thrombospondin-1 (TSP-1) TNP-470 Transforming growth factor-beta (TGF-b) Vasculostatin Vasostatin (calreticulin fragment) ZD6126 ZD 6474 farnesyl transferase inhibitors (FTI) bisphosphonates Antimitotic agents: allocolchicine Halichondrin B colchicine colchicine derivative dolstatin 10 maytansine rhizoxin thiocolchicine trityl cysteine Others: Isoprenylation inhibitors: Dopaminergic neurotoxins: 1-methyl-4-phenylpyridinium ion Cell cycle inhibitors: Staurosporine Actinomycins: Actinomycin D Dactinomycin Bleomycins: Bleomycin A2 Bleomycin B2 Peplomycin Anthracyclines: Daunorubicin Doxorubicin (adriamycin) Idarubicin Epirubicin Pirarubicin Zorubicin Mitoxantrone MDR inhibitors: Verapamil Ca.sup.2+ATPase inhibitors: Thapsigargin

Additional anti-cancer agents that may be used in the methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

### REFERENCES

ADDIN EN.REFLIST 1. Herrmann, J. and A. Lerman, The Endothelium—the Cardiovascular Health Barometer. Herz, 2008. 33(5): p. 343-353.
2. Hamel, E., Perivascular nerves and the regulation of cerebrovascular tone. J Appl Physiol, 2006. 100(3): p. 1059-64.
3. Saenz de Tejada, I., et al., Pathophysiology of erectile dysfunction. J Sex Med, 2005. 2(1): p. 26-39.
4. Provis, J. M., et al., Anatomy and development of the macula: specialisation and the vulnerability to macular degeneration. Clin Exp Optom, 2005. 88(5): p. 269-81.
5. Izikki, M., et al., Role for dysregulated endothelium—derived FGF2 signaling in progression of pulmonary hypertension. Rev Mal Respir, 2008. 25(9): p. 1192.
6. Pautler, E. L., The possible role and treatment of deficient microcirculation regulation in age-associated memory impairment. Med Hypotheses, 1994. 42(6): p. 363-6.
7. McCarron, R. M., et al., Endothelial-mediated regulation of cerebral microcirculation. J Physiol Pharmacol, 2006. 57 Suppl 11: p. 133-44.
8. Nowak, J. Z., Age-related macular degeneration (AMD): pathogenesis and therapy. Pharmacol Rep, 2006. 58(3): p. 353-63.
9. Chai, S. J., E. Barrett-Connor, and A. Gamst, Small-vessel lower extremity arterial disease and erectile dysfunction: The Rancho Bernardo study. Atherosclerosis, 2009. 203(2): p. 620-5.
10. Tuder, R. M. and J. H. Yun, Vascular endothelial growth factor of the lung: friend or foe. Curr Opin Pharmacol, 2008. 8(3): p. 255-60.
11. Kelm, M., Flow-mediated dilatation in human circulation: diagnostic and therapeutic aspects. Am J Physiol Heart Circ Physiol, 2002. 282(1): p. H1-5.
12. Celermajer, D. S., et al., Non-invasive detection of endothelial dysfunction in children and adults at risk of atherosclerosis. Lancet, 1992. 340(8828): p. 1111-5.
13. Palmer, R. M., A. G. Ferrige, and S. Moncada, Nitric oxide release accounts for the biological activity of endothelium-derived relaxing factor. Nature, 1987. 327(6122): p. 524-6.
14. Ahlers, B. A., et al., An age-related decline in endothelial function is not associated with alterations in L-arginine transport in humans. J Hypertens, 2004. 22(2): p. 321-7.
15. Taddei, S., et al., Aging and endothelial function in normotensive subjects and patients with essential hypertension. Circulation, 1995. 91(7): p. 1981-7.
16. Andrawis, N., D. S. Jones, and D. R. Abernethy, Aging is associated with endothelial dysfunction in the human forearm vasculature. J Am Geriatr Soc, 2000. 48(2): p. 193-8.
17. Ghiadoni, L., et al., Endothelial dysfunction and oxidative stress in chronic renal failure. J Nephrol, 2004. 17(4): p. 512-9.
18. Bilsborough, W., et al., Anti-tumour necrosis factor-alpha therapy over conventional therapy improves endothelial function in adults with rheumatoid arthritis. Rheumatol Int, 2006. 26(12): p. 1125-31.
19. Roifman, I., et al., Evidence of endothelial dysfunction in patients with inflammatory bowel disease. Clin Gastroenterol Hepatol, 2009. 7(2): p. 175-82.
20. Hurks, R., et al., Early endothelial dysfunction in young type 1 diabetics. Eur J Vase Endovasc Surg, 2009. 37(5): p. 611-5.
21. Crisby, M., et al., Circulating levels of autoantibodies to oxidized low-density lipoprotein and C-reactive protein levels correlate with endothelial function in resistance arteries in men with coronary heart disease. Heart Vessels, 2009. 24(2): p. 90-5.
22. Dede, D. S., et al., Assessment of endothelial function in Alzheimer's disease: is Alzheimer's disease a vascular disease? J Am Geriatr Soc, 2007. 55(10): p. 1613-7.
23. Chong, A. Y., et al., Endothelial dysfunction and damage in congestive heart failure: relation of flow-mediated dilation to circulating endothelial cells, plasma indexes of endothelial damage, and brain natriuretic peptide. Circulation, 2004. 110(13): p. 1794-8.
24. Poredos, P., Endothelial dysfunction in the pathogenesis of atherosclerosis. Int Angiol, 2002. 21(2): p. 109-16.
25. Listi, F., et al., PECAM-1/CD31 in infarction and longevity. Ann N Y Acad Sci, 2007. 1100: p. 132-9.
26. Ballard, V. L. and J. M. Edelberg, Targetsfor regulating angiogenesis in the ageing endothelium. Expert Opin Ther Targets, 2007. 11(11): p. 1385-99.
27. Lu, C., et al., Effect of age on vascularization during fracture repair. J Orthop Res, 2008. 26(10): p. 1384-9.
28. Rivard, A., et al., Age-dependent defect in vascular endothelial growth factor expression is associated with reduced hypoxia-inducible factor 1 activity. J Biol Chem, 2000. 275(38): p. 29643-7.
29. Basak, G. W., et al., Human embryonic stem cells hemangioblast express HLA-antigens. J Transl Med, 2009. 7: p. 27.
30. Stump, M. M., et al., Endothelium Grown from Circulating Blood on Isolated Intravascular Dacron Hub. Am J Pathol, 1963. 43: p. 361-7.
31. Asahara, T., et al., Isolation of putative progenitor endothelial cells for angiogenesis. Science, 1997. 275(5302): p. 964-7.
32. Takahashi, T., et al., Ischemia- and cytokine-induced mobilization of bone marrow-derived endothelial progenitor cells for neovascularization. Nat Med, 1999. 5(4): p. 434-8.
33. Asahara, T., et al., Bone marrow origin of endothelial progenitor cells responsible for postnatal vasculogenesis in physiological and pathological neovascularization. Circ Res, 1999. 85(3): p. 221-8.
34. Peichev, M., et al., Expression of VEGFR-2 and AC133 by circulating human CD34(+) cells identifies a population of functional endothelial precursors. Blood, 2000. 95(3): p. 952-8.
35. Korbling, M., et al., Recombinant human granulocyte-colony-stimulating factor-mobilized and apheresis-collected endothelial progenitor cells: a novel blood cell componentfor therapeutic vasculogenesis. Transfusion, 2006. 46(10): p. 1795-802.
36. Timmermans, F., et al., Endothelial outgrowth cells are not derived from CD133+ cells or CD45+ hematopoietic precursors. Arterioscler Thromb Vasc Biol, 2007. 27(7): p. 1572-9.
37. Rehman, J., et al., Peripheral blood “endothelial progenitor cells” are derived from monocyte/macrophages and secrete angiogenic growth factors. Circulation, 2003. 107(8): p. 1164-9.
38. Rohde, E., et al., Blood monocytes mimic endothelial progenitor cells. Stem Cells, 2006. 24(2): p. 357-67.
39. Foteinos, G., et al., Rapid endothelial turnover in atherosclerosis-prone areas coincides with stem cell repair in apolipoprotein E-deficient mice. Circulation, 2008. 117(14): p. 1856-63.
40. Werner, N., et al., Intravenous transfusion of endothelial progenitor cells reduces neointima formation after vascular injury. Circ Res, 2003. 93(2): p. e17-24.
41. Wassmann, S., et al., Improvement of endothelial function by systemic transfusion of vascular progenitor cells. Circ Res, 2006. 99(8): p. e74-83.
42. Edelberg, J. M., et al., Young adult bone marrow-derived endothelial precursor cells restore aging-impaired cardiac angiogenic function. Circ Res, 2002. 90(10): p. E89-93.
43. Ceradini, D. J. and G. C. Gurtner, Homing to hypoxia: HIF-1 as a mediator of progenitor cell recruitment to injured tissue. Trends Cardiovasc Med, 2005. 15(2): p. 57-63.
44. Schomig, K., et al., Interleukin-8 is associated with circulating CD133+ progenitor cells in acute myocardial infarction. Eur Heart J, 2006. 27(9): p. 1032-7.
45. Shintani, S., et al., Mobilization of endothelial progenitor cells in patients with acute myocardial infarction. Circulation, 2001. 103(23): p. 2776-9.
46. Andrassy, M., et al., High-mobility group box-1 in ischemia-reperfusion injury of the heart. Circulation, 2008. 117(25): p. 3216-26.
47. Palumbo, R., et al., Cells migrating to sites of tissue damage in response to the danger signal HMGB1 require NF-kappaB activation. J Cell Biol, 2007. 179(1): p. 33-40.
48. Gill, M., et al., Vascular trauma induces rapid but transient mobilization of VEGFR2(+)AC133(+) endothelial precursor cells. Circ Res, 2001. 88(2): p. 167-74.
49. Wojakowski, W., et al., Mobilization of CD34/CXCR4+, CD34/CD117+, c-met+ stem cells, and mononuclear cells expressing early cardiac, muscle, and endothelial markers into peripheral blood in patients with acute myocardial infarction. Circulation, 2004. 110(20): p. 3213-20.
50. Massa, M., et al., Increased circulating hematopoietic and endothelial progenitor cells in the early phase of acute myocardial infarction. Blood, 2005. 105(1): p. 199-206.
51. Chang, L. T., et al., Role of stromal cell-derived factor-1alpha, level and value of circulating interleukin-10 and endothelial progenitor cells in patients with acute myocardial infarction undergoing primary coronary angioplasty. Circ J, 2009. 73(6): p. 1097-104.
52. Sobrino, T., et al., The increase of circulating endothelial progenitor cells after acute ischemic stroke is associated with good outcome. Stroke, 2007. 38(10): p. 2759-64.
53. Dunac, A., et al., Neurological and functional recovery in human stroke are associated with peripheral blood CD34+ cell mobilization. J Neurol, 2007. 254(3): p. 327-32.
54. Yip, H. K., et al., Level and value of circulating endothelial progenitor cells in patients after acute ischemic stroke. Stroke, 2008. 39(1): p. 69-74.
55. Wu, J., et al., Intravenously administered bone marrow cells migrate to damaged brain tissue and improve neural function in ischemic rats. Cell Transplant, 2008. 16(10): p. 993-1005.
56. Chen, Z. Z., et al., Beneficial effect of autologous transplantation of bone marrow stromal cells and endothelial progenitor cells on cerebral ischemia in rabbits. Neurosci Lett, 2008. 445(1): p. 36-41.
57. Deng, Y. B., et al., Intravenously administered BMSCs reduce neuronal apoptosis and promote neuronal proliferation through the release of VEGF after stroke in rats. Neurol Res, 2009.
58. Ware, L. B. and M. A. Matthay, The acute respiratory distress syndrome. N Engl J Med, 2000. 342(18): p. 1334-49.
59. Suratt, B. T., et al., Human pulmonary chimerism after hematopoietic stem cell transplantation. Am J Respir Crit Care Med, 2003. 168(3): p. 318-22.
60. Yamada, M., et al., Increased circulating endothelial progenitor cells in patients with bacterial pneumonia: evidence that bone marrow derived cells contribute to lung repair. Thorax, 2005. 60(5): p. 410-3.
61. Burnham, E. L., et al., Increased circulating endothelial progenitor cells are associated with survival in acute lung injury. Am J Respir Crit Care Med, 2005. 172(7): p. 854-60.
62. Lam, C. F., et al., Autologous transplantation of endothelial progenitor cells attenuates acute lung injury in rabbits. Anesthesiology, 2008. 108(3): p. 392-401.
63. Hatada, T., et al., Plasma concentrations and importance of High Mobility Group Box protein in the prognosis of organ failure in patients with disseminated intravascular coagulation. Thromb Haemost, 2005. 94(5): p. 975-9.
64. Becchi, C., et al., The increase of endothelial progenitor cells in the peripheral blood: a new parameter for detecting onset and severity of sepsis. Int J Immunopathol Pharmacol, 2008. 21(3): p. 697-705.
65. Liu, Y., S. D. Song, and H. X. Wang, [A clinical study of the serum vascular endothelial growth factor in patients with severe sepsis]. Zhongguo Wei Zhong Bing Ji Jiu Yi Xue, 2009. 21(3): p. 172-4.
66. Matsuda, N. and Y. Hattori, Vascular biology in sepsis: pathophysiological and therapeutic significance of vascular dysfunction. J Smooth Muscle Res, 2007. 43(4): p. 117-37.
67. Regnault, V. and B. Levy, Recombinant activated protein C in sepsis: endothelium protection or endothelium therapy? Crit Care, 2007. 11(1): p. 103.
68. Rafat, N., et al., Increased circulating endothelial progenitor cells in septic patients: correlation with survival. Crit Care Med, 2007. 35(7): p. 1677-84.
69. Hristov, M., et al., Adult progenitor cells in vascular remodeling during atherosclerosis. Biol Chem, 2008. 389(7): p. 837-44.
70. Zhao, Q., et al., Stem/progenitor cells in liver injury repair and regeneration. Biol Cell, 2009. 101(10): p. 557-71.
71. Sun, Y., Myocardial repair/remodelling following infarction: roles of local factors. Cardiovasc Res, 2009. 81(3): p. 482-90.
72. Ogami, M., et al., Telomere shortening in human coronary artery diseases. Arterioscler Thromb Vase Biol, 2004. 24(3): p. 546-50.
73. Goldschmidt-Clermont, P. J., Loss of bone marrow-derived vascular progenitor cells leads to inflammation and atherosclerosis. Am Heart J, 2003. 146(4 Suppl): p. 55-12.
74. Spyridopoulos, I., et al., Telomere gap between granulocytes and lymphocytes is a determinant for hematopoetic progenitor cell impairment in patients with previous myocardial infarction. Arterioscler Thromb Vase Biol, 2008. 28(5): p. 968-74.
75. Griese, D. P., et al., Isolation and transplantation of autologous circulating endothelial cells into denuded vessels and prosthetic grafts: implications for cell-based vascular therapy. Circulation, 2003. 108(21): p. 2710-5.
76. Liu, F., et al., Transplanted endothelial progenitor cells ameliorate carbon tetrachloride-induced liver cirrhosis in rats. Liver Transpl, 2009. 15(9): p. 1092-100.
77. Nakamura, T., et al., Significance and therapeutic potential of endothelial progenitor cell transplantation in a cirrhotic liver rat model. Gastroenterology, 2007. 133(1): p. 91-107 el.
78. Xin, Z., et al., Different biological properties of circulating and bone marrow endothelial progenitor cells in acute myocardial infarction rats. Thorac Cardiovasc Surg, 2008. 56(8): p. 441-8.
79. Vila, V., et al., Inflammation, endothelial dysfunction and angiogenesis markers in chronic heart failure patients. Int J Cardiol, 2008. 130(2): p. 276-7.
80. von Haehling, S., et al., Inflammatory biomarkers in heart failure revisited: much more than innocent bystanders. Heart Fail Clin, 2009. 5(4): p. 549-60.
81. Stenvinkel, P., Inflammation in end-stage renal disease—a fire that burns within. Contrib Nephrol, 2005. 149: p. 185-99.
82. Porazko, T., et al., IL-18 is involved in vascular injury in end-stage renal disease patients. Nephrol Dial Transplant, 2009. 24(2): p. 589-96.
83. Nakae, H., et al., Involvement of IL-18 and soluble fas in patients with postoperative hepatic failure. Eur Surg Res, 2003. 35(2): p. 61-6.
84. Yumoto, E., et al., Serum gamma-interferon-inducing factor (IL-18) and IL-10 levels in patients with acute hepatitis and fulminant hepatic failure. J Gastroenterol Hepatol, 2002. 17(3): p. 285-94.
85. Petrovic-Rackov, L. and N. Pejnovic, Clinical significance of IL-18, IL-15, IL-12 and TNF-alpha measurement in rheumatoid arthritis. Clin Rheumatol, 2006. 25(4): p. 448-52.
86. Leach, S. T., et al., Local and systemic interleukin-18 and interleukin-18-binding protein in children with inflammatory bowel disease. Inflamm Bowel Dis, 2008. 14(1): p. 68-74.
87. Miles, E. A., et al., Age-related increases in circulating inflammatory markers in men are independent of BMI, blood pressure and blood lipid concentrations. Atherosclerosis, 2008. 196(1): p. 298-305.
88. Krabbe, K. S., M. Pedersen, and H. Bruunsgaard, Inflammatory mediators in the elderly. Exp Gerontol, 2004. 39(5): p. 687-99.
89. Svoboda, P., et al., Neopterin, a marker of immune response, and 8-hydroxy-2′-deoxyguanosine, a marker of oxidative stress, correlate at high age as determined by automated simultaneous high-performance liquid chromatography analysis of human urine. Anal Biochem, 2008. 383(2): p. 236-42.
90. Blasko, I., et al., Cognitive deterioration in Alzheimer's disease is accompanied by increase of plasma neopterin. J Psychiatr Res, 2007. 41(8): p. 694-701.
91. Capri, M., et al., The genetics of human longevity. Ann N Y Acad Sci, 2006. 1067: p. 252-63.
92. Ventura, E., et al., Homocysteine and inflammation as main determinants of oxidative stress in the elderly. Free Radic Biol Med, 2008.
93. van Leuven, S. I., et al., ApoAI-phosphatidylcholine infusion neutralizes the atherothrombotic effects of C-reactive protein in humans. J Thromb Haemost, 2008.
94. Nagaoka, T., et al., C-reactive protein inhibits endothelium-dependent nitric oxide-mediated dilation of retinal arterioles via enhanced superoxide production. Invest Ophthalmol Vis Sci, 2008. 49(5): p. 2053-60.
95. Butovsky, O., et al., Induction and blockage of oligodendrogenesis by differently activated microglia in an animal model of multiple sclerosis. J Clin Invest, 2006. 116(4): p. 905-15.
96. Pickering, M. and J. J. O'Connor, Pro-inflammatory cytokines and their effects in the dentate gyrus. Prog Brain Res, 2007. 163: p. 339-54.
97. Pluchino, S., et al., Persistent inflammation alters the function of the endogenous brain stem cell compartment. Brain, 2008. 131(Pt 10): p. 2564-78.
98. Fiorito, C., et al., Antioxidants increase number of progenitor endothelial cells through multiple gene expression pathways. Free Radic Res, 2008. 42(8): p. 754-62.
99. Ablin, J. N., et al., Effect of anti-TNFalpha treatment on circulating endothelial progenitor cells (EPCs) in rheumatoid arthritis. Life Sci, 2006. 79(25): p. 2364-9.
100. Bosello, S., et al., TNF-alpha blockade induces a reversible but transient effect on endothelial dysfunction in patients with long-standing severe rheumatoid arthritis. Clin Rheumatol, 2008. 27(7): p. 833-9.
101. Harley, C. B., A. B. Futcher, and C. W. Greider, Telomeres shorten during ageing of human fibroblasts. Nature, 1990. 345(6274): p. 458-60.
102. Maier, J. A., et al., Extension of the life-span of human endothelial cells by an interleukin-1 alpha antisense oligomer. Science, 1990. 249(4976): p. 1570-4.
103. Schnabl, B., et al., Replicative senescence of activated human hepatic stellate cells is accompanied by a pronounced inflammatory but less fibrogenic phenotype. Hepatology, 2003. 37(3): p. 653-64.
104. Maier, J. A., M. Statuto, and G. Ragnotti, Senescence stimulates U937-endothelial cell interactions. Exp Cell Res, 1993. 208(1): p. 270-4.
105. Shelton, D. N., et al., Microarray analysis of replicative senescence. Curr Biol, 1999. 9(17): p. 939-45.
106. Parkinson, E. K., et al., Replicative senescence as a barrier to human cancer. Biochem Soc Trans, 2000. 28(2): p. 226-33.
107. Satoh, H., et al., Telomere shortening in peripheral blood cells was related with aging but not with white blood cell count. Jpn J Hum Genet, 1996. 41(4): p. 413-7.
108. Metcalfe, J. A., et al., Accelerated telomere shortening in ataxia telangiectasia. Nat Genet, 1996. 13(3): p. 350-3.
109. Chang, E. and C. B. Harley, Telomere length and replicative aging in human vascular tissues. Proc Natl Acad Sci USA, 1995. 92(24): p. 11190-4.
110. Caplan, B. A. and C. J. Schwartz, Increased endothelial cell turnover in areas of in vivo Evans Blue uptake in the pig aorta. Atherosclerosis, 1973. 17(3): p. 401-17.
111. Erusalimsky, J. D. and D. J. Kurz, Cellular senescence in vivo: its relevance in ageing and cardiovascular disease. Exp Gerontol, 2005. 40(8-9): p. 634-42.
112. Satoh, M., et al., Effect of intensive lipid-lowering therapy on telomere erosion in endothelial progenitor cells obtained from patients with coronary artery disease. Clin Sci (Lond), 2009. 116(11): p. 827-35.
113. Farhat, N., et al., Stress-induced senescence predominates in endothelial cells isolated from atherosclerotic chronic smokers. Can J Physiol Pharmacol, 2008. 86(11): p. 761-9.
114. Kim, K. S., et al., Interferon-gamma induces cellular senescence through p53-dependent DNA damage signaling in human endothelial cells. Mech Ageing Dev, 2009. 130(3): p. 179-88.
115. Mezzano, D., et al., Inflammation, not hyperhomocysteinemia, is related to oxidative stress and hemostatic and endothelial dysfunction in uremia. Kidney Int, 2001. 60(5): p. 1844-50.
116. Satoh, M., et al., Association between oxidative DNA damage and telomere shortening in circulating endothelial progenitor cells obtained from metabolic syndrome patients with coronary artery disease. Atherosclerosis, 2008. 198(2): p. 347-53.
117. Phillips, T., et al., A dietary supplement attenuates IL-6 and CRP after eccentric exercise in untrained males. Med Sci Sports Exerc, 2003. 35(12): p. 2032-7.
118. Regensteiner, J. G., et al., Oral L-arginine and vitamins E and C improve endothelial function in women with type 2 diabetes. Vasc Med, 2003. 8(3): p. 169-75.
119. Kalani, R., et al., Effects of caloric restriction and exercise on age-related, chronic inflammation assessed by C-reactive protein and interleukin-6. J Gerontol A Biol Sci Med Sci, 2006. 61(3): p. 211-7.
120. Colbert, L. H., et al., Physical activity, exercise, and inflammatory markers in older adults: findings from the Health, Aging and Body Composition Study. J Am Geriatr Soc, 2004. 52(7): p. 1098-104.
121. Thijssen, D. H., et al., Vascular adaptations to 8-week cycling training in older men. Acta Physiol (Oxf), 2007. 190(3): p. 221-8.
122. Abidov, M., et al., Effect of Blueberin on fasting glucose, C-reactive protein and plasma aminotransferases, in female volunteers with diabetes type 2: double-blind, placebo controlled clinical study. Georgian Med News, 2006(141): p. 66-72.
123. Alexopoulos, N., et al., The acute effect of green tea consumption on endothelial function in healthy individuals. Eur J Cardiovasc Prev Rehabil, 2008. 15(3): p. 300-5.
124. Ridker, P. M., et al., Rosuvastatin to prevent vascular events in men and women with elevated C-reactive protein. N Engl J Med, 2008. 359(21): p. 2195-207.
125. Spiel, A. O., et al., Simvastatin and rosuvastatin mobilize Endothelial Progenitor Cells but do not prevent their acute decrease during systemic inflammation. Thromb Res, 2008. 123(1): p. 108-13.
126. Thum, T., et al., Suppression of endothelial progenitor cells in human coronary artery disease by the endogenous nitric oxide synthase inhibitor asymmetric dimethylarginine. J Am Coll Cardiol, 2005. 46(9): p. 1693-701.
127. Singh, S., et al., Stem cells improve left ventricular function in acute myocardial infarction. Clin Cardiol, 2009. 32(4): p. 176-80.
128. Martin-Rendon, E., et al., Autologous bone marrow stem cells to treat acute myocardial infarction: a systematic review. Eur Heart J, 2008. 29(15): p. 1807-18.
129. Abdel-Latif, A., et al., Adult bone marrow-derived cells for cardiac repair: a systematic review and meta-analysis. Arch Intern Med, 2007. 167(10): p. 989-97.
130. Rota, M., et al., Bone marrow cells adopt the cardiomyogenic fate in vivo. Proc Natl Acad Sci USA, 2007. 104(45): p. 17783-8.
131. Kajstura, J., et al., Bone marrow cells differentiate in cardiac cell lineages after infarction independently of cellfusion. Circ Res, 2005. 96(1): p. 127-37.
132. Psaltis, P. J., et al., Concise review: mesenchymal stromal cells: potential for cardiovascular repair. Stem Cells, 2008. 26(9): p. 2201-10.
133. Norol, F., et al., GFP-transduced CD34+ and Lin-CD34−hematopoietic stem cells did not adopt a cardiac phenotype in a nonhuman primate model of myocardial infarct. Exp Hematol, 2007. 35(4): p. 653-61.
134. Devanesan, A. J., et al., Endothelial progenitor cells as a therapeutic option in peripheral arterial disease. Eur J Vasc Endovasc Surg, 2009. 38(4): p. 475-81.
135. Jujo, K., M. Ii, and D. W. Losordo, Endothelial progenitor cells in neovascularization of infarcted myocardium. J Mol Cell Cardiol, 2008. 45(4): p. 530-44.
136. Jin, F., et al., Degradation of BM SDF-1 by MMP-9: the role in G-CSF-induced hematopoietic stem/progenitor cell mobilization. Bone Marrow Transplant, 2008. 42(9): p. 581-8.
137. Carion, A., et al., Stromal-derived factor 1 and matrix metalloproteinase 9 levels in bone marrow and peripheral blood of patients mobilized by granulocyte colony-stimulating factor and chemotherapy. Relationship with mobilizing capacity of haematopoietic progenitor cells. Br J Haematol, 2003. 122(6): p. 918-26.
138. Ratajczak, M. Z., et al., A pivotal role of activation of complement cascade (CC) in mobilization of hematopoietic stem/progenitor cells (HSPC). Adv Exp Med Biol, 2008. 632: p. 47-60.
139. Lee, H. M., et al., Impaired mobilization of hematopoietic stem/progenitor cells in C5-deficient mice supports the pivotal involvement of innate immunity in this process and reveals novel promobilization effects of granulocytes. Leukemia, 2009.
140. Pitchford, S. C., et al., Differential mobilization of subsets of progenitor cells from the bone marrow. Cell Stem Cell, 2009. 4(1): p. 62-72.
141. Ince, H., et al., Prevention of left ventricular remodeling with granulocyte colony-stimulating factor after acute myocardial infarction: final 1-year results of the Front-Integrated Revascularization and Stem Cell Liberation in Evolving Acute Myocardial Infarction by Granulocyte Colony-Stimulating Factor (FIRSTLINE-AMI) Trial. Circulation, 2005. 112(9 Suppl): p. I73-80.
142. Zohlnhofer, D., et al., Stem cell mobilization by granulocyte colony-stimulating factor in patients with acute myocardial infarction: a randomized controlled trial. JAMA, 2006. 295(9): p. 1003-10.
143. Hill, J. M. and J. Bartunek, The end of granulocyte colony-stimulating factor in acute myocardial infarction? Reaping the benefits beyond cytokine mobilization. Circulation, 2006. 113(16): p. 1926-8.
144. Suzuki, K., et al., Effect of granulocyte colony-stimulating factor treatment at a low dose but for a long duration in patients with coronary heart disease. Circ J, 2006. 70(4): p. 430-7.
145. Leone, A. M., et al., Usefulness of granulocyte colony-stimulating factor in patients with a large anterior wall acute myocardial infarction to prevent left ventricular remodeling (the rigenera study). Am J Cardiol, 2007. 100(3): p. 397-403.
146. Napoli, R., et al., Acute effects of growth hormone on vascular function in human subjects. J Clin Endocrinol Metab, 2003. 88(6): p. 2817-20.
147. Napoli, R., et al., Growth hormone corrects vascular dysfunction in patients with chronic heart failure. J Am Coll Cardiol, 2002. 39(1): p. 90-5.
148. Devin, J. K., et al., Low-dose growth hormone administration mobilizes endothelial progenitor cells in healthy adults. Growth Horm IGF Res, 2008. 18(3): p. 253-63.
149. Thum, T., et al., Age-dependent impairment of endothelial progenitor cells is corrected by growth-hormone-mediated increase of insulin-like growth-factor-1. Circ Res, 2007. 100(3): p. 434-43.
150. Cramer, D. E., et al., Mobilization of hematopoietic progenitor cells by yeast-derived beta-glucan requires activation of matrix metalloproteinase-9. Stem Cells, 2008. 26(5): p. 1231-40.
151. Lopez, P., et al., Treg-inducing membrane vesicles from Bifidobacterium bifidum LMG13195 as potential adjuvants in immunotherapy. Vaccine, 2012. 30(5): p. 825-9.
152. Jang, S. O., et al., Asthma Prevention by Lactobacillus Rhamnosus in a Mouse Model is Associated With CD4(+)CD25(+)Foxp3(+) T Cells. Allergy Asthma Immunol Res, 2012. 4(3): p. 150-6.
153. Qiu, X., et al., Faecalibacterium prausnitzii upregulates regulatory T cells and anti-inflammatory cytokines in treating TNBS-induced colitis. J Crohns Colitis, 2013. 7(11): p. e558-68.
154. Atarashi, K., et al., Treg induction by a rationally selected mixture of Clostridia strains from the human microbiota. Nature, 2013. 500(7461): p. 232-6.
155. Yoshida, T., et al., An increased number of CD4+CD25+ cells induced by an oral administration of Lactobacillus plantarum NRIC0380 are involved in antiallergic activity. Int Arch Allergy Immunol, 2013. 162(4): p. 283-9.
156. Khailova, L., et al., Lactobacillus rhamnosus GG improves outcome in experimental Pseudomonas aeruginosa pneumonia: potential role of regulatory T cells. Shock, 2013. 40(6): p. 496-503.
157. Narushima, S., et al., Characterization of the 17 strains of regulatory T cell-inducing human-derived Clostridia. Gut Microbes, 2014. 5(3): p. 333-9.
158. Kim, H. J., et al., Effects of Lactobacillus rhamnosus on allergic march model by suppressing Th2, Th17, and TSLP responses via CD4(+)CD25(+)Foxp3(+) Tregs. Clin Immunol, 2014. 153(1): p. 178-86.
159. Zheng, B., et al., Bifidobacterium breve attenuates murine dextran sodium sulfate-induced colitis and increases regulatory T cell responses. PLoS One, 2014. 9(5): p. e95441.
160. Liu, Y., et al., Lactobacillus reuteri DSM 17938 differentially modulates effector memory T cells and Foxp3+ regulatory T cells in a mouse model of necrotizing enterocolitis. Am J Physiol Gastrointest Liver Physiol, 2014. 307(2): p. G177-86.
161. Zhou, D., et al., Oral administration of a select mixture of Bacillus probiotics generates Trl cells in weaned F4ab/acR−pigs challenged with an F4+ ETEC/VTEC/EPEC strain. Vet Res, 2015. 46: p. 95.
162. Thakur, B. K., et al., Live and heat-killed probiotic Lactobacillus casei Lbs2 protects from experimental colitis through Toll-like receptor 2-dependent induction of T-regulatory response. Int Immunopharmacol, 2016. 36: p. 39-50.
163. Wang, K., et al., Lactobacillus casei regulates differentiation of Th17/Treg cells to reduce intestinal inflammation in mice. Can J Vet Res, 2017. 81(2): p. 122-128.
164. Fu, L., et al., Lactic acid bacteria-specific induction of CD4(+)Foxp3(+)T cells ameliorates shrimp tropomyosin-induced allergic response in mice via suppression of mTOR signaling. Sci Rep, 2017. 7(1): p. 1987.
165. Savino, F., et al., Regulatory T cells and Toll-like receptor 2 and 4 mRNA expression in infants with colic treated with Lactobacillus reuteri DSMI7938. Benef Microbes, 2018. 9(6): p. 917-925.
166. Albert, M. H., et al., Antigen-dependent suppression of alloresponses by Foxp3-induced regulatory T cells in transplantation. Eur J Immunol, 2005. 35(9): p. 2598-607.
167. Rezvani, K., et al., High donor FOXP3-positive regulatory T-cell (Treg) content is associated with a low risk of GVHD following HLA-matched allogeneic SCT. Blood, 2006. 108(4): p. 1291-7.
168. Zhai, Z., et al., Correlation of the CD4+CD25high T-regulatory cells in recipients and their corresponding donors to acute GVHD. Transpl Int, 2007. 20(5): p. 440-6.
169. Arimoto, K., et al., FOXP3 expression in peripheral blood rapidly recovers and lacks correlation with the occurrence of graft-versus-host disease after allogeneic stem cell transplantation. Int J Hematol, 2007. 85(2): p. 154-62.
170. Wolf, D., et al., Regulatory T-cells in the graft and the risk of acute graft-versus-host disease after allogeneic stem cell transplantation. Transplantation, 2007. 83(8): p. 1107-13.
171. Giorgini, A. and A. Noble, Blockade of chronic graft-versus-host disease by alloantigen-induced CD4+CD25+Foxp3+ regulatory T cells in nonlymphopenic hosts. J Leukoc Biol, 2007. 82(5): p. 1053-61.
172. Hasegawa, H., et al., Therapeutic effect of CXCR3-expressing regulatory T cells on liver, lung and intestinal damages in a murine acute GVHD model. Gene Ther, 2008. 15(3): p. 171-82.
173. Zhao, D., et al., In vivo-activated CD103+CD4+ regulatory T cells ameliorate ongoing chronic graft-versus-host disease. Blood, 2008. 112(5): p. 2129-38.
174. Magenau, J. M., et al., Frequency of CD4(+)CD25(hi)FOXP3(+) regulatory T cells has diagnostic and prognostic value as a biomarker for acute graft-versus-host-disease. Biol Blood Marrow Transplant, 2010. 16(7): p. 907-14.
175. Koreth, J., et al., Interleukin-2 and regulatory T cells in graft-versus-host disease. N Engl J Med, 2011. 365(22): p. 2055-66.
176. Hannon, M., et al., Infusion of clinical-grade enriched regulatory T cells delays experimental xenogeneic graft-versus-host disease. Transfusion, 2014. 54(2): p. 353-63.
177. Theil, A., et al., Adoptive transfer of allogeneic regulatory T cells into patients with chronic graft-versus-host disease. Cytotherapy, 2015. 17(4): p. 473-86.
178. Brunstein, C. G., et al., Umbilical cord blood-derived T regulatory cells to prevent GVHD: kinetics, toxicity profile, and clinical effect. Blood, 2016. 127(8): p. 1044-51.
179. Le Texier, L., et al., Autophagy-dependent regulatory T cells are critical for the control of graft-versus-host disease. JCI Insight, 2016. 1(15): p. e86850.
180. Chen, Y. B., et al., Increased Foxp3(+)Helios(+) Regulatory T Cells and Decreased Acute Graft-versus-Host Disease after Allogeneic Bone Marrow Transplantation in Patients Receiving Sirolimus and RGI-2001, an Activator of Invariant Natural Killer T Cells. Biol Blood Marrow Transplant, 2017. 23(4): p. 625-634.
181. Ranjan, S., et al., Activated protein C protects from GvHD via PAR2/PAR3 signalling in regulatory T-cells. Nat Commun, 2017. 8(1): p. 311.
182. Kasahara, H., et al., Generation of allo-antigen-specific induced Treg stabilized by vitamin C treatment and its application for prevention of acute graft versus host disease model. Int Immunol, 2017. 29(10): p. 457-469.
183. Riegel, C., et al., Efficient treatment of murine acute GvHD by in vitro expanded donor regulatory T cells. Leukemia, 2019.
184. Kim, B., et al., Protective effects of Bacillus probiotics against high-fat diet-induced metabolic disorders in mice. PLoS One, 2018. 13(12): p. e0210120.
185. Morita, Y., et al., Long-term intake of Lactobacillus paracasei KW3110 prevents age-related chronic inflammation and retinal cell loss in physiologically aged mice. Aging (Albany NY), 2018. 10(10): p. 2723-2740.
186. Kim, D. E., et al., Lactobacillus plantarum NK3 and Bifidobacterium longum NK49 Alleviate Bacterial Vaginosis and Osteoporosis in Mice by Suppressing NF-kappaB-Linked TNF-alpha Expression. J Med Food, 2019. 22(10): p. 1022-1031.
187. Niemann-Jonsson, A., et al., Increased rate of apoptosis in intimal arterial smooth muscle cells through endogenous activation of TNF receptors. Arterioscler Thromb Vase Biol, 2001. 21(12): p. 1909-14.
188. Chandra, D., et al., Role of aldose reductase in TNF-alpha-induced apoptosis of vascular endothelial cells. Chem Biol Interact, 2003. 143-144: p. 605-12.
189. Kubica, J., et al., Combined periprocedural evaluation of CRP and TNF-alpha enhances the prediction of clinical restenosis and major adverse cardiac events in patients undergoing percutaneous coronary interventions. Int J Mol Med, 2005. 16(1): p. 173-80.
190. Burke, J. G., et al., Human nucleus pulposis can respond to a pro-inflammatory stimulus. Spine (Phila Pa 1976), 2003. 28(24): p. 2685-93.
191. Lin, Y., et al., Propionibacterium acnes Induces Intervertebral Disc Degeneration by Promoting iNOS/NO and COX-2/PGE2 Activation via the ROS-Dependent NF-kappaB Pathway. Oxid Med Cell Longev, 2018. 2018: p. 3692752.
192. Chen, Z., et al., Overview: the role of Propionibacterium acnes in nonpyogenic intervertebral discs. Int Orthop, 2016. 40(6): p. 1291-8.
193. Yuan, Y., et al., Histological Identification of Propionibacterium acnes in Nonpyogenic Degenerated Intervertebral Discs. Biomed Res Int, 2017. 2017: p. 6192935.
194. Mottin, V. H. M. and E. S. Suyenaga, An approach on the potential use of probiotics in the treatment of skin conditions: acne and atopic dermatitis. Int J Dermatol, 2018. 57(12): p. 1425-1432.
195. Zhang, C., et al., Gut microbiota dysbiosis in male patients with chronic traumatic complete spinal cord injury. J Transl Med, 2018. 16(1): p. 353.
196. Wallace, D. J., et al., Spinal cord injury and the human microbiome: beyond the brain-gut axis. Neurosurg Focus, 2019. 46(3): p. E11.
197. Zhang, C., et al., Dysbiosis of gut microbiota is associated with serum lipid profiles in male patients with chronic traumatic cervical spinal cord injury. Am J Transl Res, 2019. 11(8): p. 4817-4834.
198. Kigerl, K. A., et al., The spinal cord-gut-immune axis as a master regulator of health and neurologicalfunction after spinal cord injury. Exp Neurol, 2019. 323: p. 113085.
199. Kigerl, K. A., et al., Gut dysbiosis impairs recovery after spinal cord injury. J Exp Med, 2016. 213(12): p. 2603-2620.
200. Kigerl, K. A., K. Mostacada, and P. G. Popovich, Gut Microbiota Are Disease-Modifying Factors After Traumatic Spinal Cord Injury. Neurotherapeutics, 2018. 15(1): p. 60-67.
201. Bajaj, J. S., et al., Altered profile of human gut microbiome is associated with cirrhosis and its complications. J Hepatol, 2014. 60(5): p. 940-7.
202. Chen, Y., et al., Gut dysbiosis in acute-on-chronic liver failure and its predictive value for mortality. J Gastroenterol Hepatol, 2015. 30(9): p. 1429-37.
203. Zhang, J. Y., et al., Interleukin-17-producing CD4(+) T cells increase with severity of liver damage in patients with chronic hepatitis B. Hepatology, 2010. 51(1): p. 81-91.
204. Zhang, G. L., et al., Imbalance of interleukin-17-producing CD4 T cells/regulatory T cells axis occurs in remission stage of patients with hepatitis B virus-related acute-on-chronic liver failure. J Gastroenterol Hepatol, 2013. 28(3): p. 513-21.
205. Zhang, G. L., et al., Increased IL-17-producing CD8(+) T cell frequency predicts short-term mortality in patients with hepatitis B virus-related acute-on-chronic liver failure. Ther Clin Risk Manag, 2018. 14: p. 2127-2136.
206. Bajaj, J. S., et al., Randomised clinical trial: Lactobacillus GG modulates gut microbiome, metabolome and endotoxemia in patients with cirrhosis. Aliment Pharmacol Ther, 2014. 39(10): p. 1113-25.
207. Saab, S., et al., Probiotics are helpful in hepatic encephalopathy: a meta-analysis of randomized trials. Liver Int, 2016. 36(7): p. 986-93.
208. Kamo, T., et al., Dysbiosis and compositional alterations with aging in the gut microbiota ofpatients with heartfailure. PLoS One, 2017. 12(3): p. e0174099.
209. Pasini, E., et al., Malnutrition and Gut Flora Dysbiosis: Specific Therapies for Emerging Comorbidities in Heart Failure. Biomed Res Int, 2015. 2015: p. 382585.
210. Yin, J., et al., Dysbiosis of Gut Microbiota With Reduced Trimethylamine-N-Oxide Level in Patients With Large-Artery Atherosclerotic Stroke or Transient Ischemic Attack. J Am Heart Assoc, 2015. 4(11).
211. Enstrom, A., et al., Detection of IL-17 and IL-23 in Plasma Samples of Children with Autism. Am J Biochem Biotechnol, 2008. 4(2): p. 114-120.
212. Blaylock, R. L. and A. Strunecka, Immune-glutamatergic dysfunction as a central mechanism of the autism spectrum disorders. Curr Med Chem, 2009. 16(2): p. 157-70.
213. Careaga, M., J. Van de Water, and P. Ashwood, Immune dysfunction in autism: a pathway to treatment. Neurotherapeutics, 2010. 7(3): p. 283-92.
214. MacFabe, D. F., et al., Effects of the enteric bacterial metabolic product propionic acid on object-directed behavior, social behavior, cognition, and neuroinflammation in adolescent rats: Relevance to autism spectrum disorder. Behav Brain Res, 2011. 217(1): p. 47-54.
215. Willette, A. A., et al., Brain enlargement and increased behavioral and cytokine reactivity in infant monkeys following acute prenatal endotoxemia. Behav Brain Res, 2011. 219(1): p. 108-15.
216. Naik, U. S., et al., A study of nuclear transcription factor-kappa B in childhood autism. PLoS One, 2011. 6(5): p. e19488.
217. Wei, H., et al., IL-6 is increased in the cerebellum of autistic brain and alters neural cell adhesion, migration and synaptic formation. J Neuroinflammation, 2011. 8: p. 52.
218. Rose, S., et al., Evidence of oxidative damage and inflammation associated with low glutathione redox status in the autism brain. Transl Psychiatry, 2012. 2: p. e134.
219. Onore, C. E., et al., Inflammatory macrophage phenotype in BTBR T+tf/J mice. Front Neurosci, 2013. 7: p. 158.
220. Noriega, D. B. and H. F. Savelkoul, Immune dysregulation in autism spectrum disorder. Eur J Pediatr, 2014. 173(1): p. 33-43.
221. de Theije, C. G., et al., Intestinal inflammation in a murine model of autism spectrum disorders. Brain Behav Immun, 2014. 37: p. 240-7.
222. Jyonouchi, H., L. Geng, and A. L. Davidow, Cytokine profiles by peripheral blood monocytes are associated with changes in behavioral symptoms following immune insults in a subset of ASD subjects: an inflammatory subtype? J Neuroinflammation, 2014. 11: p. 187.
223. Pramparo, T., et al., Prediction of autism by translation and immune/inflammation coexpressed genes in toddlers from pediatric community practices. JAMA Psychiatry, 2015. 72(4): p. 386-94.
224. Moieni, M., et al., Inflammation impairs social cognitive processing: A randomized controlled trial of endotoxin. Brain Behav Immun, 2015. 48: p. 132-8.
225. Tonhajzerova, I., et al., Inflammatory Activity in Autism Spectrum Disorder. Adv Exp Med Biol, 2015. 861: p. 93-8.
226. Akintunde, M. E., et al., Increased production of IL-17 in children with autism spectrum disorders and co-morbid asthma. J Neuroimmunol, 2015. 286: p. 33-41.
227. Tsilioni, I., et al., Children with autism spectrum disorders, who improved with a luteolin-containing dietary formulation, show reduced serum levels of TNF and IL-6. Transl Psychiatry, 2015. 5: p. e647.
228. Choi, G. B., et al., The maternal interleukin-17a pathway in mice promotes autism-like phenotypes in offspring. Science, 2016. 351(6276): p. 933-9.
229. Simsek, S., et al., Elevated levels of tissue plasminogen activator and E-selectin in male children with autism spectrum disorder. Autism Res, 2016. 9(12): p. 1241-1247.
230. Inga Jacome, M. C., et al., Peripheral Inflammatory Markers Contributing to Comorbidities in Autism. Behav Sci (Basel), 2016. 6(4).
231. Bjorklund, G., et al., Immune dysfunction and neuroinflammation in autism spectrum disorder. Acta Neurobiol Exp (Wars), 2016. 76(4): p. 257-268.
232. Prata, J., et al., Bridging Autism Spectrum Disorders and Schizophrenia through inflammation and biomarkers—pre-clinical and clinical investigations. J Neuroinflammation, 2017. 14(1): p. 179.
233. Nadeem, A., et al., Activation of IL-17 receptor leads to increased oxidative inflammation in peripheral monocytes of autistic children. Brain Behav Immun, 2018. 67: p. 335-344.
234. Osokine, I. and A. Erlebacher, Inflammation and Autism: From Maternal Gut to Fetal Brain. Trends Mol Med, 2017. 23(12): p. 1070-1071.
235. Dipasquale, V., et al., Neuroinflammation in Autism Spectrum Disorders: Role of High Mobility Group Box 1 Protein. Int J Mol Cell Med, 2017. 6(3): p. 148-155.
236. Basheer, S., et al., Immune aberrations in children with Autism Spectrum Disorder: a case-control study from a tertiary care neuropsychiatric hospital in India. Psychoneuroendocrinology, 2018. 94: p. 162-167.
237. Nadeem, A., et al., Oxidative and inflammatory mediators are upregulated in neutrophils of autistic children: Role of IL-17A receptor signaling. Prog Neuropsychopharmacol Biol Psychiatry, 2019. 90: p. 204-211.
238. Prata, J., et al., The Contribution of Inflammation to Autism Spectrum Disorders: Recent Clinical Evidence. Methods Mol Biol, 2019. 2011: p. 493-510.
239. Nadeem, A., et al., Differential regulation of NrJ2 is linked to elevated inflammation and nitrative stress in monocytes of children with autism. Psychoneuroendocrinology, 2019. 113: p. 104554.
240. Ashwood, P., et al., Spontaneous mucosal lymphocyte cytokine profiles in children with autism and gastrointestinal symptoms: mucosal immune activation and reduced counter regulatory interleukin-10. J Clin Immunol, 2004. 24(6): p. 664-73.
241. Melamed, I. R., et al., A pilot study of high-dose intravenous immunoglobulin 5% for autism: Impact on autism spectrum and markers of neuroinflammation. Autism Res, 2018. 11(3): p. 421-433.
242. Williams, B. L., et al., Impaired carbohydrate digestion and transport and mucosal dysbiosis in the intestines of children with autism and gastrointestinal disturbances. PLoS One, 2011. 6(9): p. e24585.
243. Mezzelani, A., et al., Environment, dysbiosis, immunity and sex-specific susceptibility: a translational hypothesis for regressive autism pathogenesis. Nutr Neurosci, 2015. 18(4): p. 145-61.
244. De Angelis, M., et al., Autism spectrum disorders and intestinal microbiota. Gut Microbes, 2015. 6(3): p. 207-13.
245. Iovene, M. R., et al., Intestinal Dysbiosis and Yeast Isolation in Stool of Subjects with Autism Spectrum Disorders. Mycopathologia, 2017. 182(3-4): p. 349-363.
246. Qiao, Y., et al., Alterations of oral microbiota distinguish children with autism spectrum disorders from healthy controls. Sci Rep, 2018. 8(1): p. 1597.
247. Liu, F., et al., The valproic acid rat model of autism presents with gut bacterial dysbiosis similar to that in human autism. Mol Autism, 2018. 9: p. 61.
248. Ma, B., et al., Altered Gut Microbiota in Chinese Children With Autism Spectrum Disorders. Front Cell Infect Microbiol, 2019. 9: p. 40.
249. Kong, X., et al., New and Preliminary Evidence on Altered Oral and Gut Microbiota in Individuals with Autism Spectrum Disorder (ASD): Implications for ASD Diagnosis and Subtyping Based on Microbial Biomarkers. Nutrients, 2019. 11(9).
250. Carissimi, C., et al., Functional analysis of gut microbiota and immunoinflammation in children with autism spectrum disorders. Dig Liver Dis, 2019. 51(10): p. 1366-1374.
251. Ribeiro, R., et al., Impact of vitamin deficiency on microbiota composition and immunomodulation: relevance to autistic spectrum disorders. Nutr Neurosci, 2019: p. 1-13.
252. Tabouy, L., et al., Dysbiosis of microbiome and probiotic treatment in a genetic model of autism spectrum disorders. Brain Behav Immun, 2018. 73: p. 310-319.
253. Aabed, K., et al., Bee pollen and propolis improve neuroinflammation and dysbiosis induced by propionic acid, a short chain fatty acid in a rodent model of autism. Lipids Health Dis, 2019. 18(1): p. 200.
254. Critchfield, J. W., et al., The potential role of probiotics in the management of childhood autism spectrum disorders. Gastroenterol Res Pract, 2011. 2011: p. 161358.
255. Wang, X., et al., Oral probiotic administration during pregnancy prevents autism-related behaviors in offspring induced by maternal immune activation via anti-inflammation in mice. Autism Res, 2019. 12(4): p. 576-588.
256. Meyn, R. E. and D. Murray, Cell cycle effects of alkylating agents. Pharmacol Ther, 1984. 24(2): p. 147-63.
257. Votaw, M. L. and F. E. Bull, Drug therapy for neoplastic disease in adults. Med Clin North Am, 1969. 53(6): p. 1265-78.
258. Dauber, L. G., Compound X given intravenously. On the history of the nitrogen mustards. N Y State J Med, 1984. 84(3 Pt 1): p. 122-4.
259. Scott, R. B., Cancer chemotherapy—the first twenty-five years. Br Med J, 1970. 4(5730): p. 259-65.
260. Carter, S. K. and L. M. Kershner, Cancer chemotherapy: What drugs are available. Med Times, 1976. 104(2): p. 154-62.
261. Brennan, M. J., A Survey of Progress in Chemotherapy. R I Med J, 1963. 46: p. 585-9.

1. A method of predicting health status in a mammal comprising the steps of: a) quantifying levels of circulating endothelial progenitor cells; b) quantifying levels of circulating regenerative cells; and c) correlating said levels of circulating endothelial progenitor cells and circulating regenerative cells with inflammatory and regenerative markers in the blood.

2. The method of claim 1, wherein said circulating endothelial progenitor cells express VEGF[1]receptor and c-met.

3. The method of claim 1, wherein said circulating endothelial progenitor cells express VEGF[1]receptor and c-met.

4. The method of claim 3, wherein said circulating endothelial progenitor cells multiply once every approximately 12-36 hours.

5. The method of claim 3, wherein said circulating endothelial progenitor cells multiply once every approximately 17-30 hours.

6. The method of claim 3, wherein said circulating endothelial progenitor cells multiply once every approximately 20-24 hours.

7. The method of claim 3, wherein said circulating endothelial progenitor cells produce interleukin 1 beta at a concentration of 1-8 picograms per million cells when stimulated with 1 ng/ml of lipopolysaccharide.

8. The method of claim 3, wherein said circulating endothelial progenitor cells produce interleukin 1 beta at a concentration of 5-7 picograms per million cells when stimulated with 1 ng/ml of lipopolysaccharide.

9. The method of claim 3, wherein said circulating endothelial progenitor cells produce at a concentration of 7 picograms per million cells when stimulated with 1 ng/ml of lipopolysaccharide.

10. The method of claim 3, wherein said circulating endothelial progenitor cells produce FGF[1]1 at a concentration of 9-88 picograms per million cells when stimulated with 1 ng/ml of lipopolysaccharide.

11. The method of claim 3, wherein said circulating endothelial progenitor cells produce interleukin 1 beta at a concentration of 30-79 picograms per million cells when stimulated with 1 ng/ml of lipopolysaccharide.

12. The method of claim 3, wherein said circulating endothelial progenitor cells produce interleukin 1 beta at a concentration of 40 picograms per million cells when stimulated with 1 ng/ml of lipopolysaccharide.

13. The method of claim 12, wherein said circulating endothelial progenitor cells express IL-3 receptor.

14. The method of claim 12, wherein said circulating endothelial progenitor cells express IL-5 receptor.

15. The method of claim 12, wherein said circulating endothelial progenitor cells express IL-8 receptor.

16. The method of claim 12, wherein said circulating endothelial progenitor cells express IL-10 receptor.

17. The method of claim 12, wherein said circulating endothelial progenitor cells express IL-13 receptor.

18. The method of claim 12, wherein said circulating endothelial progenitor cells express IL-17 receptor.

19. The method of claim 12, wherein said circulating endothelial progenitor cells express IL-21 receptor.

20. The method of claim 12, wherein said circulating endothelial progenitor cells express IL-33 receptor.
